(19) United States
(12) Patent Application Publication
(10) Pub. No.: US 2025/0206795 A1
(43) Pub. Date: Jun. 26, 2025

(54) FUSION PROTEINS FOR TREATING CNS DISEASES

(71) Applicant: ICHILOV TECH LTD., Tel Aviv (IL)

(72) Inventors: Naftali STERN, Nir Zvi (IL); Yossi ANIS, Tel Aviv (IL); Oren BOGIN, Tel Aviv (IL); Esther OSHER, Rishon LeZion (IL)

(73) Assignee: ICHILOV TECH LTD., Tel Aviv (IL)

(21) Appl. No.: 18/547,503

(22) PCT Filed: Feb. 22, 2022
(86) PCT No.: PCT/IL2022/050207

§ 371 Date: Aug. 22, 2023

Related U.S. Application Data
(60) Provisional application No. 63/152,345, filed on Feb. 23, 2021.

(51) Int. Cl. C07K 14/535; A61K 38/00; A61P 25/28; C07K 14/48; C07K 14/79; C12N 9/24

(52) U.S. Cl. CPC C07K 14/535; A61P 25/28; C07K 14/48; C07K 14/79; C12N 9/2402; C12Y 302/01052; A61K 38/00; C07K 2319/50; C07K 2319/74

(57) ABSTRACT

The present invention relates to fusion proteins comprising a proteinaceous therapeutic agent. Particularly, the present invention relates to fusion proteins comprising a two-domain carrier protein linked via a peptide linker to a proteinaceous therapeutic agent, wherein the two-domain carrier protein comprises a transferrin receptor binding peptide linked to granulocyte colony stimulating factor (G-CSF), pharmaceutical compositions comprising same and uses thereof for treating CNS diseases.

This application is a 371 filing of International Patent Application No. PCT/IL2022/050207 filed Feb. 22, 2022, which claims the benefit of U.S. Application No. 63/152,345 filed Feb. 23, 2021.

### INCORPORATION BY REFERENCE OF A SEQUENCE LISTING XML

A Sequence Listing is provided herewith as a Sequence Listing entitled “SECOND REVISED SEQUENCE LISTING” created on Jan. 21, 2025 and having a size of 97,416 bytes. The contents of this Sequence Listing are incorporated by reference herein in their entirety.

### FIELD OF THE INVENTION

The present invention relates to fusion proteins. Particularly, the present invention relates to fusion proteins comprising a two-domain carrier protein linked via a peptide linker to a proteinaceous therapeutic agent, wherein the two-domain carrier protein comprises a transferrin receptor binding peptide linked to granulocyte colony stimulating factor (G-CSF), the fusion proteins capable of crossing the blood brain barrier and transporting the proteinaceous therapeutic agent into, near or onto cells of the CNS, thereby treating CNS diseases, including lysosomal storage diseases.

### BACKGROUND OF THE INVENTION

Lysosomal storage diseases (LSDs) are inherited disorders characterized by the accumulation of undigested or partially digested macromolecules, which ultimately results in cellular dysfunction and clinical abnormalities. Lysosomal storage diseases result from gene mutations in lysosomal enzymes, resulting in accumulation of the enzyme substrates in lysosomes. Organomegaly, connective-tissue and ocular pathology, and central nervous system dysfunction are often associated with lysosomal storage diseases.

Over 50 lysosomal storage diseases have been described. Lysosomal storage diseases are generally classified by the accumulated substrate and include the sphingolipidoses, oligosaccharidoses, mucolipidoses, mucopolysaccharidoses (MPSs), lipoprotein storage disorders, lysosomal transport defects, neuronal ceroid lipofuscinoses and others.

Juvenile or Late-Onset Tay-Sachs (LOTS) diseases are lysosomal storage disease variants of Tay-Sachs which are much less common than the infantile form of the disease. Juvenile or late-onset Tay-Sachs diseases affect children or adults, respectively, rather than infants, and are manifested as a progressive loss of function of the nervous system. The enzyme defect resides in the alpha subunit of β-hexosaminidase A (HEXA). People with juvenile or late-onset Tay-Sachs disease have some minor residual β-hexosaminidase A activity rather than a complete absence of the active enzyme as occurs in infantile Tay-Sachs disease.

The onset of symptoms in juvenile Tay-Sachs patients is between two and ten years of age. The onset of symptoms in LOTS patients is usually between adolescence and the mid-30's, with much variation among individuals. Neurological manifestations of these disease variants include muscle weakness and neurogenic muscle atrophy, cramping, wasting, and twitching; lack of coordination; slurred speech; and dystonia. Eventually paraparesis and partial/complete dependence on others in activities of daily living evolve. Some juvenile or LOTS patients have reduced intellectual functions, which may involve memory impairment and difficulty with comprehension. Behavioral alterations can include short attention span and changes in personality. About 40% of LOTS patients exhibit psychiatric symptoms such as psychotic episodes, depression or bipolar disorders.

Therapy for LSDs includes enzyme replacement therapy to replace the disease mutant enzyme. Enzyme replacement therapy (ERT) may be applicable for peripheral manifestations of LSDs. However, ERT has been largely unsuccessful in improving central nervous system manifestations of LSDs, due to difficulty in penetrating the blood-brain barrier.

The blood-brain barrier (BBB) is a microvascular barrier between blood and brain which is made up of a capillary endothelial layer surrounded by a basement membrane and tightly associated accessory cells (pericytes, astrocytes). The brain capillary endothelium is much less permeable to low-molecular weight solutes than other capillary endothelia due to an apical band of tight association between the membranes of adjoining endothelial cells, referred to as tight junctions. In addition to diminished passive diffusion, brain capillary endothelia also exhibit less fluid-phase pinocytosis than other endothelial cells. Brain capillaries possess few fenestrae and few endocytic vesicles, compared to the capillaries of other organs. There is little transit across the BBB of large, hydrophilic molecules aside from some specific proteins such as transferrin, lactoferrin, and low-density lipoproteins, which are taken up by receptor-mediated endocytosis.

The blood-brain barrier also impedes access of beneficial active agents (e.g., therapeutic drugs and diagnostic agents) to central nervous system (CNS) tissues, necessitating the use of carriers for their transit. Indeed, management of the neurological manifestations of LSDs is significantly impeded by the inability of therapeutic enzymes to gain access to brain cell lysosomes.

WO 2007/091250 to some of the inventors of the present invention discloses a chimeric protein for the delivery of a therapeutic enzyme across the blood brain barrier, the chimeric protein comprising a protein hormone covalently linked to a therapeutic enzyme, wherein the protein hormone is able to cross the blood brain barrier; and the therapeutic enzyme is an enzyme whose deficiency is linked to a lysosomal storage disease. According to WO 2007/091250, the protein hormone can be leptin or granulocyte colony stimulating factor (G-CSF).

U.S. Pat. No. 7,943,733 discloses compositions and methods of transferrin-based fusion proteins that demonstrate a high-level expression of transferrin-based fusion proteins by inserting a helical linker between two protein domains. According to U.S. Pat. No. 7,943,733, the first protein domain can be a carrier protein such as transferrin, serum albumin, an antibody, or sFv, and the second protein domain can be a therapeutic protein such as colony stimulating factor (CSF) such as G-CSF, interferon, a cytokine, a hormone, and the like.

U.S. Pat. No. 7,956,158 discloses a polypeptide comprising a first protein domain, a second protein domain, and a dithiocyclopeptide spacer containing at least one protease cleavage site. Also disclosed are methods of producing the polypeptide and delivering the protein domains into a cell. According to U.S. Pat. No. 7,956,158, the first protein domain may be a G-CSF of about 20 kDa, and the second protein domain may be a transferrin domain of 80 kDa.

U.S. Pat. No. 8,188,032 discloses a polypeptide comprising a G-CSF domain operably linked to a transferrin domain, wherein the ability of the polypeptide to be transported into a cell expressing a transferrin receptor gene or the ability of the polypeptide to be transported across a cell expressing a transferrin receptor gene is higher than that of the G-CSF domain alone. U.S. Pat. No. 8,188,032 further discloses methods of enhancing transport of G-CSF into or across a gastrointestinal (GI) epithelial cell or methods of enhancing production of circulating neutrophils in a subject comprising administering to the subject said polypeptide, the subject may be undergoing chemotherapy for cancer, or is suffering from severe chronic neutropenia.

U.S. Pat. No. 8,785,597 discloses a fusion protein of a mutant G-CSF protein and a carrier protein, wherein the GSF-mutant protein comprises multipoint substitutions, and wherein the carrier protein can be human serum albumin, human transferrin, or antibody Fc fragment. According to U.S. Pat. No. 8,785,597, the fusion protein has longer half-life than natural G-CSF and higher G-CSF-induced biological activity in stimulating the proliferation of neutrophilic granulocytes, and can be used for treating neutropenia.

U.S. Pat. No. 10,479,822 disclose a fusion protein in which transferrin is peptide-bonded to a terminal of a granulocyte-colony stimulating factor (G-CSF) protein or a mutant G-CSF protein in which Threonine¹¹⁶ is substituted with cysteine. According to U.S. Pat. No. 10,479,822, the G-CSF mutant or the fusion protein thereof display G-CSF increased specific activity and blood stability, therefore can be used for treating ischemic diseases or neutropenia.

U.S. Pat. No. 10,759,864 discloses anti-human transferrin receptor antibody or an analog thereof which comprises specific amino acid sequences of CDR1, CDR2, and CDR3 of the heavy chain variable region, which antibody is capable of penetrating the BBB. U.S. Pat. No. 10,759,864 further discloses fusion proteins of the anti-human transferrin receptor antibody and a protein which is a lysosomal enzyme, among which β-hexosaminidase A and β-hexosaminidase B are listed.

There still remains an unmet need for improved means and methods for delivering proteinaceous therapeutic agents to the central nervous system of patients having CNS diseases, thereby treating or attenuating the progression of the CNS diseases in these patients.

### SUMMARY OF THE INVENTION

The present invention provides fusion proteins comprising a two-domain carrier protein and a proteinaceous therapeutic agent linked thereto via a peptide linker, wherein the two-domain carrier protein comprises a transferrin receptor binding peptide linked to granulocyte colony stimulating factor (G-CSF), and wherein the fusion protein is capable of passing through the blood brain barrier and transporting the proteinaceous therapeutic agent into, near or onto cells of the central nervous system (CNS) of a subject having a CNS disease. The present invention further provides polynucleotides encoding said fusion proteins, expression vectors comprising the polynucleotides, host cells comprising the expression vectors, and pharmaceutical compositions comprising same. The present invention further provides methods for treating CNS diseases comprising administering said pharmaceutical composition to a subject in need of such treatment.

The present invention discloses for the first time that intravenous administration of a fusion protein to a mouse model of Tay-Sachs, wherein the fusion protein comprises a two-domain carrier protein linked to the α subunit of β-hexosaminidase A (HEXA), and wherein the two-domain carrier protein comprises a transferrin receptor binding peptide linked to G-CSF; such administration resulted not only in the detection of the fusion protein in brain cells, specifically in the lysosomes of brain cells, but more importantly, reduced the content of GM2 ganglioside in the brain by about 60% as compared to the content of GM2 ganglioside in the brain of untreated Tay-Sachs mice. Thus, the fusion protein of the present invention crossed the blood brain barrier and restored or replenished the deficient/defective HEXA in the brain of these mice.

The present invention further discloses that the hydrolysis of GM2 ganglioside in the brain of Tay-Sachs mice by the fusion protein of the present invention was higher than that obtained by a fusion protein comprising the transferrin receptor binding peptide linked to HEXA or by a fusion protein comprising G-CSF linked to HEXA. It is shown herein that the hydrolysis of GM2 ganglioside in the brain of Tay-Sachs mice by the fusion protein of the present invention was not only additive, but rather synergistic. Without being bound to any theory or mechanism of action, it is suggested that the two-domain carrier protein enables binding of the fusion protein to transferrin receptors and/or G-CSF receptors present on the membrane of endothelial cells of the capillaries of the blood brain barrier (BBB), and by virtue of this dual BBB entry domain (i.e., transferrin receptor binding peptide and G-CSF), utilizing dual gates, the transport of the protein of interest, and specifically the therapeutic lysosomal enzyme, into the brain is facilitated.

The present invention further discloses that a peptide linker linking the two-domain carrier protein and the lysosomal enzyme, e.g., HEXA, was found to be necessary in order to express the fusion protein and to endow it with an enzymatic activity. While in the absence of a peptide linker or in the presence of a peptide linker having a rigid α helical structure attributed to (Glu-Ala-Ala-Ala-Ala-Lys) repeats, the fusion proteins were hardly expressible, and if expressible, were found to be essentially inactive in hydrolytic activity, fusion proteins comprising a peptide linker having a flexible structure attributed to (Gly-Ser) repeats, were both expressible and active in degrading GM2 ganglioside. Also, fusion proteins comprising a rigid α helical peptide linker which further comprises the amino acid sequence of an angiotensin converting enzyme (ACE) cleavage site, such fusion proteins were shown to be inactive in degrading GM2 ganglioside. However, fusion proteins comprising a flexible peptide linker attributed to (Gly-Ser) repeats which further comprises the amino acid sequence of an angiotensin converting enzyme (ACE) cleavage site, such fusion proteins were shown to be highly expressible and active in degrading GM2 ganglioside.

Thus, the fusion proteins of the present invention are significantly more effective than known fusion proteins in crossing the blood brain barrier and in replenishing HEXA activity in the brain. The present invention therefore provides an improved therapeutic means for treating lysosomal storage diseases, particularly Tay-Sachs disease, to which therapeutic agents are currently unavailable.

It is now further disclosed that a fusion protein comprising a neurotrophic factor, e.g., brain derived neurotrophic factor (BDNF), and a two-domain carrier protein linked thereto via a peptide linker, wherein the two-domain carrier protein comprises a transferrin receptor binding peptide and G-CSF; such a fusion protein was effectively expressed and exhibited neurotrophic activity. The neurotrophic activity of the fusion protein comprising BDNF was essentially identical to that of free, unlinked BDNF.

The present invention further discloses that the transferrin receptor binding peptide can be linked, directly or indirectly, to the N-terminus or C-terminus of G-CSF to form the two-domain carrier protein of the fusion protein of the invention. Yet, this variation in the structure of the two-domain carrier protein does not affect the biological activity of the proteinaceous agent.

It is further disclosed that the two-domain carrier protein can be linked via a peptide linker to the N-terminus, or more importantly to the C-terminus, of the proteinaceous therapeutic agent of the fusion protein of the invention. This is particularly critical for fusion proteins comprising neurotrophic factors. As many of the neurotrophic factors are synthetized as precursor proteins of which the N-terminal pro-domain is enzymatically cleaved and released to yield the mature active neurotrophic factor, the linking of the two-domain carrier protein, via a peptide linker, to the C-terminus of the neurotrophic factor enables releasing the N-terminal pro-domain, thus obtaining an active neurotrophic factor.

The present invention therefore provides highly advantageous fusion proteins comprising proteinaceous therapeutic agents. Due to the fact that the biological activity of the proteinaceous therapeutic agent is maintained and due to the fact that their delivery through the BBB into the CNS is facilitated, the present invention provides improved means and methods for treating CNS diseases.

According to a first aspect, the present invention provides a fusion protein comprising: a two-domain carrier protein and a proteinaceous therapeutic agent linked thereto via a peptide linker,
wherein the two-domain carrier protein comprises a transferrin receptor binding peptide linked to granulocyte colony stimulating factor (G-CSF), and
wherein the fusion protein is capable of passing through the blood brain barrier, thereby transporting the proteinaceous therapeutic agent into, near or onto cells of the central nervous system (CNS) of a subject having a CNS disease.

According to some embodiments, the proteinaceous therapeutic agent is an enzyme which is decreased, absent or mutated in the cells of the CNS.

According to additional embodiments, the fusion protein is capable of replenishing the enzyme in the cells of the CNS at a higher level than a fusion protein comprising the transferrin receptor binding peptide linked to said enzyme and/or at a higher level than a fusion protein comprising the G-CSF linked to the enzyme.

According to further embodiments, the enzyme is selected from the group consisting of lysosomal enzymes, amyloid beta (Aβ) degrading enzymes, insulin degrading enzyme, and active precursors or fragments thereof. Each possibility represents a separate embodiment of the invention.

According to yet further embodiments, the lysosomal enzyme is selected from the group consisting of β-hexosaminidase A, aspartylglucosaminidase, acid lipase, α-galactosidase A, acid ceramidase, α-L-fucosidase, α-D-mannosidase, β-D-mannosidase, arylsulphatase A, neuraminidase, α-N-acetylglucosaminidase, phosphotransferase, phosphotransferase γ-subunit, L-iduronidase, iduronate-2-sulphatase, heparan-N-sulphatase, acetylCoA:N-acetyltransferase, N-acetylglucosamine 6-sulphatase, galactose 6-sulphatase, β-galactosidase, N-acetylgalactosamine 4-sulphatase, hyalurono-glucosaminidase, multiple sulphatases, palmitoyl protein thioesterase, tripeptidyl peptidase I, acid sphingomyelinase, cathepsin K, α-galactosidase B, and active precursors or fragments thereof. Each possibility represents a separate embodiment of the invention.

According to still further embodiments, the lysosomal enzyme is β-hexosaminidase A or an active fragment thereof. According to a certain embodiment, the lysosomal enzyme is the α subunit of β-hexosaminidase A, or an active fragment thereof.

According to another embodiment, the amyloid beta degrading enzyme is neuronal α-amylase.

According to some embodiments, the proteinaceous therapeutic agent of the fusion protein is a neurotrophic factor.

According to additional embodiments, the neurotrophic factor is selected from the group consisting of brain derived neurotrophic factor (BDNF), nerve growth factor (NGF), glial derived neurotrophic factor (GDNF), neurotrophins (NTs), neurturin, neuregulin, netrin, and ciliary neurotrophic factor (CNTF). Each possibility represents a separate embodiment of the invention. According to an exemplary embodiment, the neurotrophic factor is BDNF.

According to some embodiments, the proteinaceous therapeutic agent of the fusion protein is an antibody or an active fragment thereof directed to growth factors or growth factor receptors secreted by or expressed on tumor cells in the brain. According to exemplary embodiments, the antibody is bevacizumab or atezolizumab.

According to some embodiments, the CNS disease is selected from the group consisting of lysosomal storage diseases, neurodegenerative diseases, and primary or metastatic brain tumors. Each possibility represents a separate embodiment of the invention.

According to additional embodiments, the lysosomal storage disease is selected from the group consisting of GM2-gangliosidosis type I/Tay-Sachs disease; GM2-gangliosidosis type II/Sandhoff disease; GM1-gangliosidosis types I/III; aspartylglucosaminuria; cystinosis; Danon disease; Fabry disease; Farber's disease; fucosidosis; galactosialidosis types I/II; Gaucher disease types 1, 2, 3; globoid cell leucodystrophy/Krabbé disease; glycogen storage disease II/Pompe disease; α-mannosidosis types I/II; β-mannosidosis; metachromatic leukodystrophy; mucolipidosis type I/sialidosis types I/II; mucolipidosis types II/III; mucolipidosis type IIIC pseudo-Hurler polydystrophy; mucopolysaccharidosis (e.g., types I, II, IIIA, IIIB, IIIC, IIID, IVA, IVB, VI, VII, IX); multiple sulphatase deficiency; neuronal ceroid lipofuscinosis; Niemann-Pick disease; Schindler disease types I/II; and sialic acid storage disease. Each possibility represents a separate embodiment of the invention. According to one exemplary embodiment, Tay-Sachs disease is juvenile Tay-Sachs disease. According to another exemplary embodiment, Tay-Sachs disease is late onset Tay-Sachs (LOTS) disease.

According to one embodiment, the neurodegenerative disease is Alzheimer's disease.

According to another embodiment, the CNS diseases is a primary brain tumor which is glioblastoma.

According to some embodiments, the fusion protein comprises a lysosomal enzyme which is the α subunit of β-hexosaminidase A comprising the amino acid sequence as set forth in any one of SEQ ID NOs:1-5, or an active fragment thereof. According to a certain embodiment, the a subunit of β-hexosaminidase A has the amino acid sequence as set forth in SEQ ID NO:1.

According to further embodiments, the fusion protein comprises the transferrin receptor binding peptide comprising the amino acid sequence as set forth in any one of SEQ ID NOs: 6-10, 20, 36, and 37, or an active analog or fragment thereof. According to exemplary embodiments, the transferrin receptor binding peptide has the amino acid sequence as set forth in any one of SEQ ID NOs:6 and 36.

According to still further embodiments, the fusion protein comprises G-CSF which comprises the amino acid sequence as set forth in any one SEQ ID NOs:11-15, or an active fragment or analog thereof. According to an exemplary embodiment, G-CSF has the amino acid sequence as set forth in SEQ ID NO:11.

According to yet further embodiments, the fusion protein comprises a peptide linker which comprises an amino acid sequence having a flexible structure. According to still further embodiments, the peptide linker comprises a peptide selected from the group consisting of: an angiotensin converting enzyme (ACE) cleavage site, a (Gly-Ser)ₘ peptide wherein m ranges from 1 to 30, a (Gly-Gly-Ser)ₙ peptide wherein n ranges from 1 to 20, a peptide having the sequence of GTGSAGSAAGSGEF (SEQ ID NO:35), an analog, fragment and combinations thereof. Each possibility represents a separate embodiment of the invention. According to a certain embodiment, the ACE cleavage site comprises the amino acid sequence as set forth in SEQ ID NO:21. According to exemplary embodiments, the peptide linker comprises the amino acid sequence as set forth in any one of SEQ ID NOs:16, 17, and 35. According to particular embodiments, the peptide linker has the amino acid sequence as set forth in SEQ ID NO:16 or 35.

According to some embodiments, the fusion protein comprises the α subunit of β-hexosaminidase A comprising the amino acid sequence as set forth in any one of SEQ ID NOs:1-5, or an active fragment thereof, and G-CSF comprising the amino acid sequence as set forth in any one SEQ ID NOs:11-15, or an active fragment or analog thereof. Each possibility represents a separate embodiment of the invention.

According to additional embodiments, the fusion protein comprises the α subunit of β-hexosaminidase A comprising the amino acid sequence as set forth in any one of SEQ ID NOs:1-5, or an active fragment thereof, the transferrin receptor binding peptide comprising the amino acid sequence as set forth in any one of SEQ ID NOs:6-10, 20, 36 and 37, or an active analog or fragment thereof, and G-CSF comprising the amino acid sequence as set forth in any one SEQ ID NOs:11-15, or an active fragment or analog thereof. Each possibility represents a separate embodiment of the invention.

According to further embodiments, the fusion protein comprises the α subunit of β-hexosaminidase A comprising the amino acid sequence as set forth in any one of SEQ ID NOs:1-5, or an active fragment thereof, the transferrin receptor binding peptide comprising the amino acid sequence as set forth in any one of SEQ ID NOs:6-10, 20, 36 and 37, or an active analog or fragment thereof, G-CSF comprising the amino acid sequence as set forth in any one SEQ ID NOs:11-15, or an active fragment or analog thereof, and the peptide linker comprising a peptide selected from the group consisting of: an angiotensin converting enzyme (ACE) cleavage site, a (Gly-Ser)ₘ peptide wherein m ranges from 1 to 30, a (Gly-Gly-Ser)ₙ peptide wherein n ranges from 1 to 20, a peptide having the sequence of GTGSAGSAAGSGEF (SEQ ID NO:35), an analog, fragment and combinations thereof. Each possibility represents a separate embodiment of the invention. According to certain embodiments, the peptide linker has the amino acid sequence as set forth in any one of SEQ ID NOs:16, 17, and 35.

According to one exemplary embodiment, the fusion protein comprises the α subunit of β-hexosaminidase A having the amino acid sequence as set forth in SEQ ID NO:1, the transferrin receptor binding peptide having the amino acid sequence as set forth in SEQ ID NO:6, G-CSF having the amino acid sequence as set forth in SEQ ID NO:11, and the peptide linker having the amino acid sequence as set forth in SEQ ID NO:16. According to a certain embodiment, the fusion protein has the amino acid sequence as set forth in SEQ ID NO:18.

According to some embodiments, the fusion protein comprises BDNF or a precursor thereof comprising the amino acid sequence as set forth in any one of SEQ ID NOs: 30 and 31, or an active fragment or analog thereof, and G-CSF comprising the amino acid sequence as set forth in any one of SEQ ID NOs:11-15, or an active fragment or analog thereof. Each possibility represents a separate embodiment of the invention.

According to further embodiments, the fusion protein comprises BDNF or a precursor thereof comprising the amino acid sequence as set forth in any one of SEQ ID NOs: 30 and 31, or an active fragment or analog thereof, G-CSF comprising the amino acid sequence as set forth in any one of SEQ ID NOs:11-15, or an active fragment or analog thereof, and transferrin receptor binding peptide comprising the amino acid sequence as set forth in any one of SEQ ID NOs:36 and 37, or an active analog or fragment thereof. Each possibility represents a separate embodiment of the invention.

According to still further embodiments, the fusion protein comprises BDNF or a precursor thereof comprising the amino acid sequence as set forth in any one of SEQ ID NOs: 30 and 31, or an active fragment or analog thereof, G-CSF comprising the amino acid sequence as set forth in any one SEQ ID NOs:11-15, or an active fragment or analog thereof, transferrin receptor binding peptide comprising the amino acid sequence as set forth in any one of SEQ ID NOs:36 and 37, or an active analog or fragment thereof, and the peptide linker comprising a peptide selected from the group consisting of: an angiotensin converting enzyme (ACE) cleavage site, a (Gly-Ser)ₘ peptide wherein m ranges from 1 to 30, a (Gly-Gly-Ser)ₙ peptide wherein n ranges from 1 to 20, a peptide having the sequence of GTGSAGSAAGSGEF (SEQ ID NO:35), an analog, fragment and combinations thereof. Each possibility represents a separate embodiment of the invention. According to a certain embodiment, the peptide linker has the amino acid sequence as set forth in SEQ ID NO:35.

According to a certain embodiment, the fusion protein comprises BDNF having the amino acid sequence as set forth in SEQ ID NO:30 or the BDNF precursor having the amino acid sequence as set forth in SEQ ID NO:31, G-CSF having the amino acid sequence as set forth in SEQ ID NO:11, transferrin receptor binding peptide having the amino acid sequence as set forth in SEQ ID NO:36, and a peptide linker having the amino acid sequence as set forth in SEQ ID NO:35. According to exemplary embodiment, the fusion protein has the amino acid sequence as set forth in SEQ ID NOs:38 or 49.

According to another aspect, the present invention provides a polynucleotide encoding the fusion protein according to the principles of the present invention. According to an exemplary embodiment, the polynucleotide has the nucleotide sequence as set forth in SEQ ID NO:40. According to additional exemplary embodiments, the polynucleotide has the nucleotide sequence as set forth in any one of SEQ ID NO:44, 47, and 48.

According to another aspect, the present invention provides an expression vector comprising the polynucleotide of the present invention.

According to another aspect, the present invention provides a host cell comprising the expression vector of the present invention.

According to another aspect, the present invention provides a pharmaceutical composition comprising as an active agent at least one of the following agents: (i) a fusion protein according to the invention; (ii) a polynucleotide encoding the fusion protein of the invention; (iii) an expression vector comprising the polynucleotide of the invention; and (iv) a host cell comprising the expression vector of the invention; and a pharmaceutically acceptable carrier.

According to some embodiments, the pharmaceutical composition is formulated for parenteral administration. According to a certain embodiment, the pharmaceutical composition is formulated for intravenous administration.

According to another aspect, the present invention provides a method of treating a CNS disease comprising administering to a subject in need of such treatment a therapeutically effective amount of a pharmaceutical composition of the present invention, thereby treating the CNS disease.

According to some embodiments, the CNS disease is selected from the group consisting of lysosomal storage diseases, neurodegenerative diseases, and primary or metastatic brain tumors. Each possibility represents a separate embodiment of the invention.

According to an exemplary embodiment, the lysosomal storage disease is GM2-gangliosidosis type I/Tay-Sachs disease. According to further embodiments, Tay-Sachs disease is juvenile or late onset Tay-Sachs (LOTS) disease. According to a certain embodiment, the fusion protein has the amino acid sequence as set forth in SEQ ID NO:18.

According to yet further embodiments, the CNS disease is a neurodegenerative disease, such as Parkinson's disease or Huntington's disease, and the fusion protein has the amino acid sequence as set forth in any one of SEQ ID NOs:38 and 49.

According to still further embodiments, the CNS disease is Alzheimer's disease and the proteinaceous therapeutic agent is an amyloid beta degrading enzyme.

According to yet further embodiments, the CNS disease is primary or metastatic brain tumor and the proteinaceous therapeutic agent is bevacizumab or atezolizumab.

According to a certain embodiment, the subject to be treated is human.

According to further embodiments, the pharmaceutical composition to be administered for treating the subject is formulated for parenteral administration. According to a certain embodiment, the pharmaceutical composition is formulated for intravenous administration.

According to yet further embodiments, the pharmaceutical composition is administered once a day, three times a week, twice a week, once a week, twice a month, or once a month, for so long as the CNS disease or at least one symptom associated therewith is treated.

According to another aspect, the present invention provides a pharmaceutical composition according to the principles of the present invention for use in treating a CNS disease.

These and other embodiments of the present invention will be better understood in relation to the description, examples and claims that follow.

### BRIEF DESCRIPTION OF THE FIGURES

FIGS. 1A-B show photomicrographs of immunofluorescence of GM2 in cultured human Tay-Sachs glia cells in the presence of Tr-G-CSF-HEXA. Cultured Tay-Sachs glia cells were incubated in the absence (control; FIG. 1A) or presence of Tr-G-CSF-HEXA having the peptide linker of SEQ ID NO:16 (FIG. 1B), and the content of GM2 in the cells was detected by anti-GM2 antibodies.

FIGS. 2A-C show photomicrographs of immunohistofluorescent detection of mouse Tr-G-CSF-HEXA in the brain of wild type mice. Mouse Tr-G-CSF-HEXA was administered intravenously to mice, and three hours, three days or six days after administration (FIGS. 2A-C, respectively), Tr-G-CSF-HEXA was detected in the mice brains by immunostaining with anti 6-Histidine antibodies (red staining); the nuclei were stained with DAPI (blue staining). As a control, wild type mice were injected with vehicle only, and three hours after injection the mice brains were subjected to immunostaining with anti 6-Histidine antibodies, and the nuclei were stained with DAPI.

FIG. 3 shows graphic representation of GM2 content in brain extracts of Tay-Sachs mice HEXA^{(-/-)} treated with either mouse G-CSF-HEXA or vehicle only. Tay-Sachs mice HEXA^{(-/-)} were injected intravenously with G-CSF-HEXA (3 nmol each injection) or vehicle only twice a week for 7 weeks, and the GM2 content in brain extracts was determined.

FIG. 4 shows graphic representation of GM2 content in brain extracts of Tay-Sachs mice HEXA^{(-/-)} treated with either mouse Tr-HEXA or vehicle only. Tay-Sachs mice HEXA^{(-/-)} were injected intravenously with Tr-HEXA (8 nmol each injection) or vehicle only three times a week for 4 weeks, and the GM2 content in brain extracts was determined.

FIG. 5 shows graphic representation of GM2 content in brain extracts of Tay-Sachs mice HEXA^{(-/-)} treated with either mouse Tr-G-CSF-HEXA or vehicle only. Tay-Sachs mice HEXA^{(-/-)} were injected intravenously with Tr-G-CSF-HEXA (3 nmol each injection) or vehicle only twice a week for 6 weeks, and the GM2 content in brain extracts was determined.

FIG. 6 shows Western blot analysis of proBDNF-G-CSF-Tr purified on Ni column. “Start” denotes unpurified medium, the medium which passed through the column, namely the unbound material, is denoted “UB”, and the imidazole eluted proBDNF-G-CSF-Tr protein peak in fractions 12-17 was combined and is denoted “F12-17”. The starting material, the unbound material and F12-17 were analyzed by Western blot using anti-His antibodies, anti-BDNF antibodies, and anti-proBDNF antibodies.

FIG. 7 shows Western blot analysis of maturation of proBDNF-G-CSF-Tr to BDNF-G-CSF-Tr. The maturation was achieved by enzymatic cleavage of 1 or 0.2 mg purified proBDNF-G-CSF-Tr with furin at 30° C. for 4 hours. As a control, 1 mg of proBDNF-G-CSF-Tr was incubated at the same conditions in the absence of furin. The digestion products were resolved on SDS PAGE, followed by Western blot analysis with anti-tetra-His antibody.

FIGS. 8A-B show Western blot analysis of MAPK phosphorylation induced by proBDNF-G-CSF-Tr or by BDNF. TRK-B stably-transfected HEK cells were stimulated with either BDNF or proBDNF-G-CSF-Tr for 5 min. The induced MAPK phosphorylation was detected by anti-phospho Tyrosine 204/Threonine 202 antibodies (FIG. 8A). Control cells were not stimulated with either protein. The amount of cell proteins in each treatment was identical as evaluated by anti Akt antibodies (FIG. 8B).

### DETAILED DESCRIPTION OF THE INVENTION

The present invention provides fusion proteins comprising a two-domain carrier protein, a peptide linker, and a proteinaceous therapeutic agent, such as a lysosomal enzyme, wherein the two-domain carrier protein is linked to the proteinaceous therapeutic agent via the peptide linker, wherein the two-domain carrier protein comprises a transferrin receptor binding peptide linked to granulocyte colony stimulating factor (G-CSF), and wherein the fusion protein is capable of passing through the blood brain barrier, thereby delivering or transporting the proteinaceous therapeutic agent into, near or onto cells of the CNS of a subject having a CNS disease. The present invention further provides isolated polynucleotides encoding the fusion proteins of the present invention, expression vectors comprising the isolated polynucleotides, host cells comprising the expression vectors, and pharmaceutical compositions comprising same. Further provided are methods for treating CNS diseases comprising administering said pharmaceutical compositions.

### Peptides and Proteins

The present invention provides fusion proteins comprising: a two-domain carrier protein which comprises an isolated transferrin receptor binding peptide linked to an isolated G-CSF, a peptide linker, and an isolated therapeutic protein, e.g., an isolated lysosomal enzyme or an isolated neurotrophic factor, wherein the two-domain carrier protein is linked via the peptide linker to the therapeutic agent.

The term “fusion protein” as used herein refers to a protein that is created through the joining of at least three polynucleotide sequences, which originally code for separate proteins, polypeptides or peptides; translation of the joined coding sequences results in a single, fusion protein, typically with functional properties derived from each of the separate polypeptides.

The term “isolated” as used herein refers to material that is substantially or essentially free from components that normally accompany it in its native state. For example, an “isolated protein” or an “isolated polynucleotide” and the like, as used herein, include the in vitro isolation and/or purification of a protein or polynucleotide molecule from its natural cellular environment, and from association with other components of the cell.

The terms “peptide”, “polypeptide” and “protein” are used interchangeably herein to refer to a polymer of amino acid residues. A protein may contain one or more polymers of amino acid residues.

The term “proteinaceous” as used herein refers to an agent formed from amino acid residues.

The term “cells” as used herein refers to neuronal cells. Yet, other cell types in the CNS, such as astrocytes, oligodendrocytes and glial cells, are included.

Transferrin is a glycoprotein found in biological fluids of vertebrates; it has a molecular weight of around 80 kDa and contains two specific high-affinity iron binding sites. Transferrin binds to iron and consequently mediates its transport through blood. When transferrin loaded with iron encounters a transferrin receptor on the surface of a cell, transferrin binds to the transferrin receptor and is transported into the cell.

As such, transferrin receptors have been shown to play a crucial role in iron transport. Two transferrin receptors have been identified in humans, transferrin receptor 1 and transferrin receptor 2. Both receptors are transmembrane glycoproteins. TfR1 is a high affinity ubiquitously expressed receptor which is regulated by intracellular iron concentration, while TfR2 is expressed in certain cell types and is unaffected by intracellular iron concentrations. TfR2 binds to transferrin with a 25-30-fold lower affinity than TfR1. It was demonstrated that brain vascular endothelial cells express transferrin receptors and that transport of transferrin across the BBB occurs via receptor-mediated vesicular transcytosis (transport of cargo from apical to basal side, or vice versa, in intracellular vesicles).

Various studies have tested the delivery of agents to the CNS using either transferrin itself, or an antibody against TfR, as a ferry for cargo with otherwise poor BBB penetration properties. However, as transferrin or an antibody against TfR are large molecules, the present inventors aimed at producing fusion proteins containing a shorter carrier protein with limited conformational constrains which includes two domains capable of binding to two different receptors on the membranes of endothelial cells of the capillaries of BBB, thereby facilitating the transport of the fusion protein through the BBB.

The fusion proteins of the present invention therefore comprise a carrier protein which comprises a transferrin receptor binding peptide and G-CSF.

The term “transferrin receptor binding peptide” as used herein refers to a peptide which binds to a transferrin receptor and is transported or internalized into a cell by the transferrin receptor. This term encompasses active fragments of transferrin and active analogs of these fragments. Yet, the full-length transferrin is excluded from the present invention. The transferrin receptor binding peptides of the present invention are typically of about 5 to about 100 amino acid long, preferably of about 5 to about 25 amino acid long, although longer peptides of up to about 150 amino acid residues may be used. Various peptides are known to bind to transferrin receptors and to be internalized, among which are the peptides having the amino acid sequence as set forth in any one of SEQ ID NO:7-10 and 37 (see, for example, Lee et al., Eur. J. Biochem. 268: 2004-2012 (2001) and Crook et al. J. Mol. Biol. 432: 3989-4009 (2020)). According to some embodiments, the transferrin receptor binding peptide comprises at its N-terminus and/or C-terminus a peptide comprising (Gly-Ser)ₚ, wherein p ranges from 1 to 15. According to certain embodiments, the transferrin receptor binding peptide comprises or consists of the amino acid sequence as set forth in any one of SEQ ID NOs:6, 20, or 36.

The terms “fragment” or “active fragment” as used herein refer to a peptide or protein having only a portion of the full-length naturally occurring protein which maintain at least 70%, 75%, 80%, 85%, 90%, 95%, or preferably 100% of the binding activity and/or biological activity of the naturally occurring protein, as determined by in vitro or in vivo tests known in the art (see, for example, the Examples herein below).

The terms “analog” or “active analog” denote a peptide or protein having greater than about 75%, 80%, 85%, 90%, 95%, or 99% identity of its amino acid sequence to the corresponding sequence of a naturally occurring peptide or protein, which maintain at least 70%, 75%, 80%, 85%, 90%, 95%, or preferably 100% of the binding activity and/or biological activity of the naturally occurring peptide or protein, as determined by in vitro or in vivo tests known in the art (see, for example, the Examples herein below).

The analog comprises an altered sequence by amino acid substitutions, additions, deletions, or chemical modifications. By using “amino acid substitutions”, it is meant that functionally equivalent amino acid residues are substituted for residues within the sequence resulting in a silent change. For example, one or more amino acid residues within the sequence can be substituted by another amino acid of a similar polarity, which acts as a functional equivalent, resulting in a silent alteration. Substitutes for an amino acid within the sequence may be selected from other members of the class to which the amino acid belongs. For example, the non-polar (hydrophobic) amino acids include alanine, leucine, isoleucine, valine, proline, phenylalanine, tryptophan and methionine. The polar neutral amino acids include glycine, serine, threonine, cysteine, tyrosine, asparagine, and glutamine. The positively charged (basic) amino acids include arginine, lysine and histidine. The negatively charged (acidic) amino acids include aspartic acid and glutamic acid. Such substitutions are known as conservative substitutions. Additionally, a non-conservative substitution may be made in an amino acid that does not contribute to the biological activity, e.g., binding activity.

The term “internalization” refers to a process in which peptides or proteins are engulfed by the cell membrane and transported into the cell. Various methods are available for detecting internalization of peptides or proteins including, but not limited to, immunohistochemical methods, fluorescent labeling, and biological activity assays (see, for example, Examples 2 and 3 herein below).

The term “about” refers to ±20% of a given value, preferably to ±10% of a given value.

Granulocyte colony stimulating factor (G-CSF) is a 19.6 kDa glycoprotein that stimulates the bone marrow to produce granulocytes and stem cells and release them into the bloodstream. G-CSF also stimulates the survival, proliferation, differentiation, and function of neutrophil precursors and mature neutrophils. It is therefore commonly used to treat neutropenia.

G-CSF has been also shown to act on neuronal cells as a neurotrophic factor. It induces neurogenesis, increases neuroplasticity, and counteracts apoptosis.

G-CSF receptors have been shown to be expressed by neurons in the brain and spinal cord. Due to its neuroprotective properties, G-CSF has been tested for use in cerebral ischemia and in other neurological diseases such as amyotrophic lateral sclerosis.

Human G-CSF is expressed as a precursor protein consisting of 207 amino acid residues (SEQ ID NO:12) or 204 amino acid residues (SEQ ID NO:14). A signal peptide consisting of 30 amino acid residues at the amino terminus is cleaved, and 177 or 174 amino acid mature proteins are produced (SEQ ID NO:13; SEQ ID NO:15, respectively). According to a certain embodiment, G-CSF corresponding to amino acid residues 30-207 of G-CSF precursor has the amino acid sequence as set forth in SEQ ID NO:11.

The term “G-CSF” as used herein refers to G-CSF protein that binds to G-CSF receptors and undergoes transport or internalization into a cell by the G-CSF receptors, and includes G-CSF protein precursors, G-CSF mature proteins, G-CSF fragments, and analogs thereof (see, for example, U.S. Pat. Nos. 8,188,032, 8,785,597, and 10,479,822, incorporated by reference as if fully set forth herein). G-CSF can optionally exhibit neuroprotective activities such as inhibition of neuronal apoptosis and/or induction of neurogenesis. The present invention encompasses human G-CSF as well as G-CSF of other mammalian species such as bovine, monkey, goat, etc.

According to the present invention, the transferrin receptor binding peptide is linked directly or via a peptide to G-CSF. Though direct linking is feasible, the inventors of the present application observed that a short peptide, such as of 2 to 30 amino acid long, preferably of 2 to 20 amino acids long, comprising glycine and serine residues, enabled the generation of biologically active fusion proteins. The transferrin receptor binding peptide can also be linked to G-CSF via a peptide having an & helical structure (see, for example, U.S. Pat. No. 7,943,733).

It is to be understood that the organization of the two domains within the carrier protein can vary, i.e., the transferrin receptor binding peptide can be linked at its carboxy terminus, directly or via a peptide linker, to the amino terminus of G-CSF, or the transferrin receptor binding peptide can be linked at its amino terminus, directly or via a peptide linker, to the carboxy terminus of G-CSF.

The present invention provides fusion proteins comprising as an active agent an enzyme selected from the group consisting of lysosomal enzymes, amyloid beta degrading enzymes, insulin degrading enzyme, and active precursors or fragments thereof.

Various lysosomal enzymes are known which, when absent, decreased or mutated are associated with lysosomal storage diseases (LSDs). The lysosomal enzymes include, but are not limited to, β-hexosaminidase A, aspartylglucosaminidase, acid lipase, α-galactosidase A, acid ceramidase, α-L-fucosidase, α-D-mannosidase, β-D-mannosidase, arylsulphatase A, neuraminidase, α-N-acetylglucosaminidase, phosphotransferase, phosphotransferase γ-subunit, L-iduronidase, iduronate-2-sulphatase, heparan-N-sulphatase, acetylCOA:N-acetyltransferase, N-acetylglucosamine 6-sulphatase, galactose 6-sulphatase, β-galactosidase, N-acetylgalactosamine 4-sulphatase, hyalurono-glucosaminidase, multiple sulphatases, palmitoyl protein thioesterase, tripeptidyl peptidase I, acid sphingomyelinase, cathepsin K, α-galactosidase B, and active fragments thereof.

According to some embodiments, the lysosomal enzyme is β-hexosaminidase A.

β-hexosaminidase A is a heterodimer composed of an alpha subunit and a beta subunit. The alpha subunit polypeptide is encoded by the HEXA gene while the beta subunit is encoded by the HEXB gene. Gene mutations in the gene encoding the beta subunit (HEXB) often result in Sandhoff disease; whereas mutations in the gene encoding the alpha subunit (HEXA) decrease the hydrolysis of G_{M2} gangliosides, which is the main cause of Tay-Sachs disease.

Human α subunit of β-hexosaminidase A is expressed as a precursor protein consisting of 529 amino acid residues as set forth in SEQ ID NO:2. A signal peptide consisting of 22 amino acid residues at the amino terminus is cleaved, leaving a 507 amino acid protein as set forth in SEQ ID NO:1. An a subunit fragment consisting of amino acids 109-529 is set forth in SEQ ID NO:3. A fragment containing amino acids 1-191 of the alpha subunit is set forth in SEQ ID NO:4. A fragment containing amino acids 403-529 of the alpha subunit is set forth in SEQ ID NO:5. It is to be noted that these fragments are active in hydrolysis of GM2 gangliosides. According to a certain embodiment, the α subunit of β-hexosaminidase A comprises or consists of the amino acid sequence as set forth in SEQ ID NO:1. As exemplified herein below, the activity of a fusion protein comprising HEXA as set forth in SEQ ID NO:1 was determined by hydrolysis of GM2 gangliosides in brains of an animal model of late-onset Tay-Sachs disease. The present invention encompasses HEXA proteins of other mammalian species, such as bovine, goat, monkey, etc.

According to some embodiments, the proteinaceous therapeutic agent is an amyloid beta degrading enzyme including, but not limited to, neuronal α-amylase.

According to some embodiments, the proteinaceous therapeutic agent is a neurotrophic factor.

The term “neurotrophic factor” refers to a protein that supports the growth, survival, and differentiation of both developing and mature neurons. The term “neurotrophic factor” includes neurotrophic factor precursors, active fragments and analogs thereof.

Most NTFs belong to one of three families: (1) neurotrophins, (2) glial cell-line derived neurotrophic factor family ligands (GFLs), and (3) neuropoietic cytokines. Neurotrophins are found in both precursor form, known as pro-neurotrophins, and in mature form. The mature forms are proteins of about 120 amino acids in length that exist in physiological states as stable, non-covalent approximately 25 kDa homodimers. Among neutrophins, brain derived neurotrophic factor (BDNF), nerve growth factor (NGF), and neutrophin-3 and 4/5 are listed.

BDNF acts on certain neurons of the central nervous system (CNS) and the peripheral nervous system (PNS), helping to support the survival of existing neurons and encourage the growth and differentiation of new neurons and synapses. Increasing BDNF has been associated with the treatment of a number of disorders including Parkinson's disease, Huntington's disease (HD), amyotrophic lateral sclerosis (ALS), Alzheimer's Disease, bipolar disorder and acute mania.

Therapy using NGF is assumed to prevent the development of Alzheimer's disease, to prevent the loss of brain function in stroke and to improve the patient's quality of life in peripheral diabetic neuropathy.

The biological effects of BDNF, NGF, and other neurotrophins are mediated by binding to cellular receptors of two classes: high affinity receptors of the tyrosine kinase family and a low affinity p75 receptor. The p75 receptor is a glycoprotein with a molecular weight of 75 kDa. It has no intrinsic catalytic activity, but is associated with the ERK family of soluble kinases and has a role in the protection of neurons against apoptosis. All the neurotrophins can bind to this receptor.

The specificity of individual neurotrophins is determined by their binding to p140^{trk}, a particular type of tyrosine kinase (Trk) receptors, with NGF and NT-3 binding to TrkA, while BDNF and NT-4/5 binding to TrkB. The binding is followed by receptor dimerization, resulting in autophosphorylation of intracellular tyrosine residues of the receptor by internal domains of the kinase. This in turn initiates a cascade of enzymatic reactions, including MAPK phosphorylation, that mediate the biological effects of neurotrophins, including an increased survival of neurons.

The term “BDNF” refers to mature BDNF, precursors, analogs, and fragments thereof. The amino acid sequence of human and mouse BDNF, a 119 amino acid protein, is set forth in SEQ ID NO:30. The amino acid sequence of human and mouse BDNF precursors, referred to also as proBDNF, is as set forth in SEQ ID NOs:31 and 32. The present invention encompasses BDNFs from hot blood mammals, including goat, monkey, bovine, mouse, etc.

Examples of other neurotrophic factors include, but are not limited to, glial derived neurotrophic factor (GDNF), neurturin, neuregulin, netrin, and ciliary neurotrophic factor (CNTF).

Proteinaceous therapeutic agents can also be antibodies or fragments thereof directed to primary or metastatic brain tumors. According to some embodiments, the antibodies or fragments thereof bind to growth factors or growth factor receptors secreted by or expressed on tumor cells in the brain, thereby attenuating or inhibiting the growth of primary brain tumors or of brain metastases.

Examples of growth factors include, but are not limited to, epidermal growth factor (EGF), transforming growth factor (TGF)-β, vascular endothelial growth factor (VEGF), and platelet-derived growth factor (PDGF).

Examples of antibodies include, but are not limited to, bevacizumab or atezolizumab.

The fusion protein of the present invention further comprises a peptide linker.

The term “peptide linker” as used herein refers to a peptide linked at its first terminal end to the two-domain carrier protein and at its second terminal end to the proteinaceous therapeutic agent. Thus, the peptide linker can be linked at its amino terminus to the two-domain carrier protein and at its carboxy terminus to the proteinaceous therapeutic agent. Alternatively, the peptide linker can be linked at its amino terminus to the proteinaceous therapeutic agent and at its carboxy terminus to the two-domain carrier protein.

According to some embodiments, the peptide linker comprises an angiotensin converting enzyme cleavage site.

The term “angiotensin converting enzyme cleavage site” or ACE-cleavage site as used herein refers to a peptide sequence which is cleavable by ACE. Various peptide sequences have been shown to be cleaved by ACE. According to a certain embodiment, the ACE-cleavage site has the amino acid sequence DRVYIHPFHL as set forth in SEQ ID NO:21, also designated Angiotensin I (see, for example, Pharm. Res. 2020, 164:105372. doi: 10.1016/j.phrs.2020.105372, and a variety of peptides disclosed therein).

According to some embodiments, the peptide linker comprises or consists of a flexible linker.

The term “flexible linker” as used herein refers to a peptide having an amino acid sequence which allows conformational flexibility of the peptides or proteins attached thereto.

As observed by the inventors of the present application, a peptide linker having an & helical structure which reduces or prevents conformational flexibility of the peptides or proteins attached thereto due to its rigid structure reduced or even eliminated the activity of the lysosomal enzymes of the fusion proteins. Thus, fusion proteins comprising a peptide linker comprising (EAAAAK) repeats as set forth in SEQ ID NO:51, the latter are known to form an α helical structure, as set forth in SEQ ID NOs:22 and 23, were found to be inactive in GM2 ganglioside hydrolysis as exemplified herein below.

Thus, according to some embodiments, the flexible linker of the present invention comprises (Gly-Ser)ₘ, wherein m ranges from 1 to 30. According to additional embodiments, the peptide linker comprises (Gly-Gly-Ser)ₙ, wherein n ranges from 1 to 20. According to some embodiments, the peptide linker comprises ACE-cleavage site, (Gly-Ser)ₘ, and (Gly-Gly-Ser)ₙ, thus having a flexible conformation. According to further embodiments, the ACE cleavage site is located at any position along the sequence of the flexible peptide linker, other than at the carboxy terminus of the peptide linker. According to certain embodiments, the peptide linker comprises or consists of the amino acid sequence as set forth in any one of SEQ ID NOs:16, 17, and 35.

It is noted that the peptides and proteins constituting the fusion proteins of the present invention are covalently linked or attached to each other by a peptide bond.

The fusion proteins of the present invention can be chemically synthesized or, preferably, produced as a recombinant protein. For production of a recombinant protein, a DNA encoding the fusion protein is constructed and transcribed to an mRNA. The mRNA is then translated to the recombinant protein. To facilitate production of the recombinant protein, a secretion signal can be added at the N-terminus of the protein. The recombinant protein can be secreted from a cell into the culture medium and can be collected and purified thereafter.

TABLE 1
List of proteins, peptides and polynucleotides
and their sequence identification.

| | SEQ |
|---|---|
| | ID |
| Proteins, peptides, and polynucleotides | NO: |
| Human β-hexosaminidase α subunit protein (23-529) | 1 |
| Human β-hexosaminidase α subunit precursor protein (1-529) | 2 |
| Human β-hexosaminidase α subunit fragment protein (89-529) | 3 |
| Human β-hexosaminidase α subunit fragment protein (1-191) | 4 |
| Human β-hexosaminidase α subunit fragment protein (403-529) | 5 |
| Transferrin receptor binding peptide with Gly-Ser-Ser-Arg linker | 6 |
| Transferrin receptor binding peptide | 7 |
| Transferrin receptor binding peptide | 8 |
| Transferrin receptor binding peptide | 9 |
| Transferrin receptor binding peptide | 10 |
| Human G-CSF protein (30-207) | 11 |
| Human G-CSF precursor protein (1-207) | 12 |
| Human G-CSF protein (31-207) | 13 |
| Human G-CSF precursor protein (1-204) | 14 |
| Human G-CSF protein (31-204) | 15 |
| Peptide linker (ACE cleavage site and Gly-Ser peptide) | 16 |
| Peptide linker (ACE cleavage site and Gly- Ser peptide) | 17 |
| Fusion protein: human Tr-GCSF-HEXA | 18 |
| Mouse G-CSF protein | 19 |
| Transferrin receptor binding peptide with a Gly-Ser linker | 20 |
| ACE cleavage site (amino acid sequence) | 21 |
| Peptide linker (ACE cleavage site and α helical structure) | 22 |
| Peptide linker (ACE cleavage site and α helical structure) | 23 |
| Mouse HEXA protein | 24 |
| Leader sequence peptide | 25 |
| TEV-6-histidine peptide | 26 |
| Fusion protein: mouse G-CSF-HEXA | 27 |
| Fusion protein: mouse Tr-HEXA | 28 |
| Fusion protein: mouse Tr-GCSF-HEXA | 29 |
| BDNF - mature protein | 30 |
| Human proBDNF | 31 |
| Mouse proBDNF | 32 |
| Furin cleavage site of proBDNF | 33 |
| Enterokinase cleavage site | 34 |
| Peptide linker | 35 |
| Transferrin receptor binding peptide with (Gly-Ser) repeats | 36 |
| Transferrin receptor binding peptide | 37 |
| Fusion protein: human BDNF-G-CSF-Tr | 38 |
| Leader sequence of proBDNF | 39 |
| Nucleotide sequence of fusion protein: human Tr-GCSF-HEXA | 40 |
| Nucleotide sequence of fusion protein: mouse G-CSF-HEXA | 41 |
| Nucleotide sequence of fusion protein: mouse Tr-HEXA | 42 |
| Nucleotide sequence of fusion protein: mouse Tr-G-CSF-HEXA | 43 |
| Nucleotide sequence of fusion protein: human BDNF-G-CSF-Tr | 44 |
| Nucleotide sequence of human/mouse BDNF | 45 |
| Nucleotide sequence of fusion protein: mouse proBDNF-G-CSF-Tr | 46 |
| Nucleotide sequence of fusion protein: mouse pro-domain human | 47 |
| BDNF-G-CSF-Tr | |
| Nucleotide sequence of fusion protein: human proBDNF-G-CSF-Tr | 48 |
| Fusion protein: human proBDNF-G-CSF-Tr | 49 |
| Fusion protein: mouse proBDNF-G-CSF-Tr | 50 |

### Polynucleotides

The present invention further provides polynucleotides encoding the fusion proteins of the invention. Such polynucleotides can be constructed using recombinant DNA technology well known in the art.

The term “polynucleotide” means a polymer of deoxyribonucleic acid (DNA) or ribonucleic acid (RNA), which can be derived from any source, can be single- or double-stranded, and can optionally contain synthetic, non-natural, or altered nucleotides, which is capable of being incorporated into DNA or RNA polymers. The term includes, for example, a recombinant DNA which is incorporated into a vector, into an autonomously replicating plasmid or virus, or into the genomic DNA of a prokaryote or eukaryote, or which exists as a separate molecule (e.g., as a cDNA or a genomic or cDNA fragment produced by PCR or restriction enzyme digestion).

The term “encoding” refers to the inherent property of specific sequences of nucleotides in an isolated polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

One who is skilled in the art will appreciate that more than one nucleic acid may encode any given fusion protein in view of the degeneracy of the genetic code and the allowance of exceptions to classical base pairing in the third position of the codon, as given by the so-called “Wobble rules.” Moreover, polynucleotides that include more or less nucleotides can result in the same or equivalent proteins. Accordingly, it is intended that the present invention encompasses all polynucleotides that encode the peptides and proteins of the present invention with their respective activity. The present invention also encompasses polynucleotides with substitutions, additions, or deletions, which direct the synthesis of fragments or analogs of the fusion proteins so that the fragments or analogs maintain the biological activity of the corresponding fusion proteins, e.g., binding to the corresponding receptor, internalization by that receptor, and enzyme activity.

Polynucleotides which encode naturally occurring G-CSF polypeptides and HEXA polypeptides are known (see, WO 2007/091270); and others can be deduced from the polypeptide sequences discussed herein. The nucleotide sequences encoding mature human/mouse BDNF, human proBDNF and mouse proBDNF are set forth in SEQ ID NOs:30, 31, and 32, respectively. According to specific embodiments, the nucleotide sequences encoding the human fusion proteins of the invention comprise or consist of any one of SEQ ID No:40, 44, and 48. According to a certain embodiment, the polynucleotide sequence is as set forth in SEQ ID NO:30.

The polynucleotides of the present invention can be expressed as a transported protein where the fusion protein is isolated from the medium in which the host cell containing the polynucleotide is grown, or can be expressed as an intracellular protein by deleting the leader or other peptides, in which case the fusion protein is isolated from the host cells. The fusion protein so isolated is then purified by protein purification methods known in the art.

Alternatively, the fusion proteins of the invention can be provided to the brain by transferring an expression vector comprising a polynucleotide sequence encoding the fusion protein to cells within the brain. The cells produce and secrete the fusion protein such that it is suitably provided to cells within the brain to replenish the lysosomal enzyme which is absent or partially active so as to attenuate or treat a lysosomal storage disease.

The terms “vector” or “expression vector” are used interchangeably throughout the specification and claims and define a nucleic acid capable of transporting another nucleic acid to which it has been linked.

The vectors must be introduced into the cells in a manner such that they are capable of expressing the isolated polynucleotide encoding the fusion protein of the invention. Any suitable vector can be so employed, many of which are known in the art. Examples of such vectors include naked DNA vectors (such as oligonucleotides or plasmids), viral vectors such as adeno-associated viral vectors, adenoviral vectors, herpes virus vectors, packaged amplicons, papilloma virus vectors, picornavirus vectors, polyoma virus vectors, retroviral vectors, SV40 viral vectors, vaccinia virus vectors, and other vectors. In addition to the expression vector of interest, the vector can also include other genetic elements, such as, for example, genes encoding a selectable marker (e.g., β-gal or a marker conferring resistance to a toxin), a pharmacologically active protein, a transcription factor, or other biologically active substance.

In addition to the polynucleotide sequences encoding the fusion proteins of the invention, the expression vectors comprise a promoter. The promoter drives the expression of the polynucleotides within the cells. Many viral promoters are appropriate for use in such an expression cassette (e.g., retroviral ITRs, LTRs, immediate early viral promoters (IEp) (such as herpes virus IEp (e.g., ICP4-IEp and ICPO-IEp) and cytomegalovirus (CMV) IEp), and other viral promoters (e.g., late viral promoters, latency-active promoters (LAPs), Rous Sarcoma Virus (RSV) promoters, and Murine Leukemia Virus (MLV) promoters). Other suitable promoters are eukaryotic promoters, which contain enhancer sequences (e.g., the rabbit β-globin regulatory elements), constitutively active promoters (e.g., the β-actin promoter, etc.), signal and/or tissue specific promoters (e.g., inducible and/or repressible promoters, the metallothionine promoter, neuron-specific promoters such as the neurofilament promoter, etc.).

Within the expression vector, the polynucleotide encoding the fusion protein of the invention and the promoter are operably linked such that the promoter is able to drive the expression of the fusion protein polynucleotide. The expression vector can optionally include other elements, such as splice sites, polyadenylation sequences, transcriptional regulatory elements (e.g., enhancers, silencers, etc.), or other sequences.

As used herein, the term “operably linked” refers to a linkage of polynucleotide elements in a functional relationship. A polynucleotide or nucleic acid sequence is “operably linked” when it is placed into a functional relationship with another polynucleotide or nucleic acid sequence. Thus, a promoter, or rather a transcription regulatory sequence, is operably linked to a coding sequence if it affects the transcription of the coding sequence. Operably linked means that the DNA sequences being linked are typically contiguous.

Methods for manipulating an expression vector comprising an isolated polynucleotide are well known in the art (see, e.g., Sambrook et al., 1989, Molecular Cloning: A Laboratory Manual, 2d edition, Cold Spring Harbor Press) and include direct cloning, site specific recombination using recombinases, homologous recombination, and other suitable methods of constructing a recombinant vector. In this manner, an expression vector can be constructed such that it can be replicated in any desired cell, expressed in any desired cell, and can even become integrated into the genome of any desired cell.

The expression vector is introduced into the cells by any means appropriate for the transfer of DNA into cells. Many such methods are well-known in the art (Sambrook et al., supra). Thus, in the case of prokaryotic cells, vector introduction may be accomplished, for example, by electroporation, transformation, transduction, conjugation, or mobilization. For eukaryotic cells, vectors may be introduced through the use of, for example, electroporation, transfection, infection, DNA coated microprojectiles, or protoplast fusion.

Various cells can be used as host cells to express the fusion proteins of the present invention. The term “host cell” refers to the particular cell into which the expression vector is introduced and to the progeny or potential progeny of such a cell.

A host cell can be any prokaryotic or eukaryotic cell. For example, a polypeptide of the invention can be expressed in bacterial cells such as E. coli, insect cells, yeast or mammalian cells (such as Chinese hamster ovary cells (CHO), HEK293 cells, a Hela cells, HT-1080 fibrosarcoma cells or COS cells). Other suitable host cells are known to those skilled in the art.

Cells into which the fusion protein polynucleotide can be transferred under the control of an inducible promoter, if necessary, can be used as transient transformants. Such cells themselves can then be transferred into a mammal for therapeutic benefit therein. The cells can be transferred to a site in the mammal such that the fusion protein expressed therein and secreted therefrom can exert its activity, i.e., lysosomal enzyme activity. Alternatively, particularly in the case of cells to which the vector has been added in vitro, the cells may first be subjected to several rounds of clonal selection (facilitated usually by the use of a selectable marker sequence in the vector) to select for stable transformants. Such stable transformants are then transferred to a mammal for therapeutic benefit therein.

The fusion protein can also be provided to the brain by transfecting into a population of other cells an expression vector comprising an isolated polynucleotide encoding a fusion protein according to the invention, whereby the fusion protein is expressed in and secreted from said other cells.

Successful expression of the polynucleotide can be assessed using standard molecular biological techniques (e.g., Northern hybridization, Western blotting, immunoprecipitation, enzyme immunoassay, etc.). Reagents for detecting the expression of the fusion protein polynucleotide and the secretion of the fusion protein from transfected cells are known in the art (see also examples herein below).

The fusion proteins produced by recombinant techniques whether secreted from the transfected cells to the medium or produced as intracellular proteins can be purified so that the fusion protein will be substantially pure when administered to a subject. The term “substantially pure” refers to a compound, e.g., a protein, which has been separated from components which accompany it. Typically, a compound is substantially pure when at least 10%, more preferably at least 20%, more preferably at least 50%, more preferably at least 60%, more preferably at least 80%, more preferably at least 90%, and most preferably at least 99% of the total material (by wet or dry weight, or by mol percent or mol fraction) in a sample is the compound of interest. Purity can be measured by any appropriate method, e.g., in the case of proteins by column chromatography such as gel filtration, affinity chromatography, HPLC, and gel electrophoresis.

### Pharmaceutical Compositions

The pharmaceutical compositions of the present invention comprise as an active agent: (i) a fusion protein according to the principles of the present invention; (ii) a polynucleotide encoding said fusion protein; (iii) an expression vector comprising said polynucleotide; and/or (iv) a host cell comprising the expression vector; and a pharmaceutically acceptable carrier.

The term “pharmaceutically acceptable” means approved by a regulatory agency of the Federal or a state government or listed in the U.S. Pharmacopeia or other generally recognized pharmacopeia for use in humans. The term “carrier” refers to a diluent, adjuvant, excipient, or vehicle with which the therapeutic compound is administered. Such pharmaceutical carriers can be sterile liquids, such as water and oils, including those of petroleum, animal, vegetable or synthetic origin, such as peanut oil, soybean oil, mineral oil, sesame oil and the like, polyethylene glycols, glycerin, propylene glycol or other synthetic solvents. Water, saline solutions, aqueous dextrose solutions, and glycerol solutions can be employed as liquid carriers, for example, for injection or infusion. Suitable pharmaceutical excipients include starch, glucose, lactose, sucrose, gelatin, sodium stearate, glycerol monostearate, sodium chloride, propylene glycol, and the like. The composition, if desired, can also contain minor amounts of wetting or emulsifying agents, or pH buffering agents such as acetates, citrates or phosphates. Preservative such as benzyl alcohol or methyl parabens; antioxidants such as ascorbic acid or sodium bisulfite; chelating agents such as ethylenediaminetetraacetic acid; and agents for the adjustment of tonicity such as sodium chloride or dextrose are also envisioned.

The compositions can take the form of solutions, suspensions, emulsions, tablets, capsules, powders, sustained-release formulations, and the like, depending on the route of administration chosen.

Routes of administration that are appropriate for practicing the present invention include parenteral route of administration. However, oral, nasal, and rectal administration routes can be practiced as well.

For parenteral administration, the pharmaceutical composition of the invention can be formulated in aqueous solutions, preferably in physiologically compatible buffers such as Hank's solution, Ringer's solution, or physiological saline buffer. Additionally, suspensions of the active compounds may be prepared as appropriate oily injection suspensions. Suitable lipophilic solvents or vehicles include fatty oils such as sesame oil, or synthetic fatty acids esters such as ethyl oleate, triglycerides or liposomes. Aqueous injection suspensions may contain substances, which increase the viscosity of the suspension, such as sodium carboxymethyl cellulose, sorbitol or dextran. Optionally, the suspension may also contain suitable solubilizers or agents, which increase the solubility of the compounds, to allow for the preparation of highly concentrated solutions. Parenteral formulations may optionally contain one or more additional ingredients, among which may be mentioned preservatives (e.g., when the formulations are presented in multi-dose containers), buffers to provide a suitable pH value for the formulation, and sodium chloride, or glycerin, to render a formulation isotonic with the blood.

For oral administration, the pharmaceutical composition of the invention can be formulated as tablets, capsules, dragees, liquids, gels, syrups, slurries, suspensions, and the like. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropyl methylcellulose, sodium carboxymethyl cellulose; and/or physiologically acceptable polymers such as polyvinylpyrrolidone (PVP). If desired, disintegrating agents may be added, such as cross-linked polyvinyl pyrrolidone, agar, or alginic acid or a salt thereof such as sodium alginate. Examples of suitable pharmaceutical carriers are described in “Remington's Pharmaceutical Sciences” by E. W. Martin.

For buccal administration, the compositions may take the form of tablets or lozenges formulated in conventional manner.

For nasal administration by inhalation, the active agents for use according to the present invention are conveniently delivered in the form of a droplet, mist or an aerosol spray presentation from a pressurized pack or a nebulizer with the use of a suitable propellant, e.g., dichlorodifluoromethane, trichlorofluoromethane, dichloro-tetrafluoroethane or carbon dioxide. In the case of a pressurized aerosol, the dosage unit may be determined by providing a valve to deliver a metered amount. Capsules and cartridges of, e.g., gelatin for use in an inhaler or insufflator may be formulated containing a powder mix of the compound and a suitable powder base such as lactose or starch.

The pharmaceutical composition of the present invention may contain additives such as buffers, isotonizing agents, preservatives, pH adjusting agents, thickeners, chelating agents, and suspending agents.

Examples of buffers include, but are not limited to, phosphate buffers (e.g., sodium dihydrogen phosphate dihydrate, etc.), carbonate buffers (e.g., sodium bicarbonate, etc.), borate buffers (e.g., borax, etc.), citrate buffers (e.g., trisodium citrate dihydrate, etc.), tartrate buffers (e.g., sodium tartrate, etc.), acetate buffers (e.g., sodium acetate, etc.), and amino acids (e.g., sodium glutamate, ε-aminocaproic acid, etc.).

Examples of isotonizing agents include, but are not limited to, saccharides such as sorbitol, glucose and mannitol; polyhydric alcohols such as glycerin, polyethylene glycol and propylene glycol; and salts such as sodium chloride.

Examples of preservatives include, but are not limited to, benzalkonium chloride, benzethonium chloride, parahydroxybenzoates (e.g., methyl parahydroxybenzoate, ethyl parahydroxybenzoate, etc.), benzyl alcohol, sorbic acid or salts thereof, thimerosal, and chlorobutanol.

Examples of pH adjusting agents include, but are not limited to, hydrochloric acid, acetic acid, phosphoric acid, sodium hydroxide, potassium hydroxide, and borax.

Examples of thickeners include, but are not limited to, hydroxyethyl cellulose, hydroxypropyl cellulose, methyl cellulose, hydroxypropyl methylcellulose, carboxymethyl cellulose and salts thereof.

Examples of chelating agents include, but are not limited to, disodium edetate, and ethylenediaminetetraacetic acid (EDTA).

Examples of suspending agents include, but are not limited to, polysorbates such as polysorbate 80.

The pharmaceutical compositions of the present invention can be formulated in an extended-release pharmaceutical dosage form as known in the art (see, for example, U.S. Pat. Nos. 6,605,303; 6,419,958; 6,245,357, the content of which is incorporated by reference as if fully set forth herein). Thus, an extended-release pharmaceutical dosage form can comprise a polymer, and optionally one or more additional pharmaceutically acceptable excipient or carrier. Polymers that can be used for the preparation of the extended-release pharmaceutical dosage form include hydrophilic polymers, hydrophobic polymers, and a combination thereof. Suitable hydrophilic polymers are, for example, hydroxypropyl methylcellulose, hydroxypropyl cellulose, ethylhydroxy ethylcellulose, hydroxyethyl cellulose, carboxymethyl cellulose, sodium carboxymethyl cellulose, methyl cellulose, polyethylene oxides, polyvinyl alcohols, tragacanth, and xanthan. These polymers can be used alone or in mixtures with each other. Hydrophobic polymers are exemplified by, for example, polyvinyl chloride, ethyl cellulose, polyvinyl acetate and acrylic acid copolymers, such as Eudragith™. The polymers can be used alone or as mixtures. The extended release pharmaceutical dosage forms can further comprise binders such as, for example, sugars, polyvinyl pyrrolidine, starches and gelatin; surfactants such as non-ionic surfactants such as, for example, polysorbate 80, or ionic surfactants such as, for example, sodium lauryl sulfate; lubricants such as, for example, magnesium stearate, sodium stearyl fumarate, or acetyl palmitate; fillers such as, for example, sodium aluminum silicate, lactose, or calcium phosphate; glidants such as, for example, talc and aerosol; and antioxidants.

### Uses

The present invention provides methods for treating a CNS disease comprising administering to a subject in need of such treatment a pharmaceutical composition according to the principles of the present invention.

The terms “treatment” and “treating”, used interchangeably throughout the specification and claims, are meant to include: (a) relieving the disease, i.e., causing regression of the disease; (b) inhibiting the disease, i.e., arresting its development (e.g., reducing the rate of disease progression); (c) relieving or reducing one or more symptoms of the disease; and/or (d) preventing the disease from occurring in a subject which may be predisposed to the disease but has not yet been diagnosed as having it.

The term “therapeutically effective amount” of the active agent means an amount of the active agent effective to abate, alleviate and/or treat an LSD. Thus, a therapeutically effective amount of the active agent can reduce or relieve the disease or one or more symptoms associated therewith in a treated subject as compared to the manifestation of the disease prior to treatment by at least 10%, 20%, 30%, 40%, 50%, 60%, 70%, 80%, 90% or by at least 95%, or by any integer in between, or can improve the condition of a treated subject to be similar to that of a healthy subject.

The subject to be treated is human, although other mammals, such as pet animals, can be treated as well.

According to some embodiments, the CNS disease is a lysosomal storage disease (LSD). LSD is selected from the group consisting of GM2-gangliosidosis type I/Tay-Sachs disease; GM2-gangliosidosis type II/Sandhoff disease; GM1-gangliosidosis types I/III; aspartylglucosaminuria; cystinosis; Danon disease; Fabry disease; Farber's disease; fucosidosis; galactosialidosis types I/II; Gaucher disease types 1, 2, 3; globoid cell leucodystrophy/Krabbé disease; glycogen storage disease II/Pompe disease; α-mannosidosis types I/II; β-mannosidosis; metachromatic leukodystrophy; mucolipidosis type I/sialidosis types I/II; mucolipidosis types II/III; mucolipidosis type IIIC pseudo-Hurler polydystrophy; mucopolysaccharidosis (e.g., types I, II, IIIA, IIIB, IIIC, IIID, IVA, IVB, VI, VII, IX); multiple sulphatase deficiency; neuronal ceroid lipofuscinosis; Niemann-Pick disease; Schindler disease types I/II; and sialic acid storage disease. According to additional embodiments, the LSD is infantile, juvenile or late-onset Tay-Sachs disease. According to a certain embodiment, Tay-Sachs disease is juvenile or late-onset Tay-Sachs disease (LOTS).

Tay-Sachs disease belongs to the subgroup of lysosomal storage diseases designated the G_{M2}-gangliosidoses which result from a failure in lysosomal degradation of G_{M2}-ganglioside. In Tay-Sachs disease, the enzyme responsible for the breakdown of G_{M2}-ganglioside, β-hexosaminidase A, is defective due to a mutation in one of the subunits making up the enzyme molecule, the α chain (HEXA). This genetically transmitted structural defect leads to functional decline in β-hexosaminidase A enzyme activity. Since the highest concentration of gangliosides is found in the central nervous system, the absence of normal β-hexosaminidase A is particularly detrimental to the brain and spinal cord.

The classic infantile form of Tay-Sachs disease, which results from very severe β-hexosaminidase A deficiency, is manifested by muscle weakness and neurogenic muscle atrophy that usually leads to death between 3 and 5 years of age. Patients with juvenile Tay-Sachs or LOTS, on the other hand, in whom there is some residual activity of β-hexosaminidase A (2-5% of the normal range), manifest in late childhood, in their teens or, occasionally, much later, signs of cerebellar damage such as impaired speech (dysarthria), abnormal gait (ataxia) and anterior horn cell malfunction, leading to proximal limb weakness and muscle atrophy. In some patients, clinical presentation and disease course may be dominated by neuropsychiatric problems such as psychotic episodes, bipolar disorder or depression.

One way to overcome the progressively detrimental sequence of cellular events induced by lysosomal enzyme deficiency is to administer a synthetic, fully functioning enzyme to replace the defective enzyme. It was shown that a small increase in intracellular enzyme activity can achieve correction of the LSD. In Tay-Sachs, it was found that only a low level of HEXA activity is needed to ameliorate the clinical phenotype of the disease, and asymptomatic individuals have been identified with residual activities of ≥10% of normal HEXA activity. There is also evidence that about 10% of normal HEXA levels represent a “critical threshold” for relieving the disease.

According to some embodiment, the CNS disease is a neurodegenerative disease. Neurodegenerative diseases include, but are not limited to, Alzheimer's disease, Parkinson's disease, amyotrophic lateral sclerosis (ALS), and Huntington's disease.

Alzheimer's disease is a progressive, degenerative disorder that attacks the brain's nerve cells, or neurons, resulting in loss of memory, thinking and language skills, and behavioral changes. Alzheimer's disease is a leading cause of death in adults. Histologically, the brain of persons afflicted with Alzheimer's disease is characterized by a distortion of the intracellular neurofibrils and the presence of senile plaques composed of granular or filamentous masses with an amyloid protein core, largely due to the accumulation of β amyloid peptide (Aβ) in the brain. Aβ accumulation plays a role in the pathogenesis and progression of the disease and is a proteolytic fragment of amyloid precursor protein (APP).

The fusion proteins of the present invention which comprise amyloid β degrading enzymes, such as neuronal α amylase or insulin degrading enzyme, can be useful for transporting these enzymes into the CNS, thereby treating Alzheimer's disease.

The fusion proteins of the present invention which comprise a neurotrophic factor can be useful for treating Parkinson's disease, amyotrophic lateral sclerosis (ALS), and Huntington's disease.

Parkinson's disease is a chronic and progressive degenerative disease of the brain that impairs motor control, speech, and other functions. One of the most striking features of Parkinson's disease is that it primarily affects a restricted neuronal population in the brain. Although other neurons are also affected, the dopaminergic neurons of the substantia nigra pars compacta are the most vulnerable to the disease process. BDNF has potent effects on survival and morphology of mesencephalic dopaminergic neurons, and thus its loss contributes to death of these cells in Parkinson's disease (PD).

Huntington's disease (HD) is a neurodegenerative disorder characterized by motor, cognitive, and psychiatric symptoms and by a progressive degeneration of neurons in basal ganglia in brain cortex. Patients suffering from HD have significantly lower BDNF levels in serum compared to healthy controls.

Amyotrophic lateral sclerosis (ALS) is a chronic and debilitating neurodegenerative disease which involves degeneration of cortical, bulbar and medullar motor neurons.

Thus, the fusion proteins of the present invention which comprise a neurotrophic factor, such as BDNF, can be useful for treating neurodegenerative diseases.

Other CNS diseases that can be treated with the pharmaceutical compositions of the invention are primary brain tumors, such as glioblastoma, or metastatic brain tumors from primary tumors, such as from lung cancer or breast cancer.

The pharmaceutical compositions of the present invention can be administered by parenteral administration route, although oral and nasal administration routes can also be used. Parenteral administration route includes intravenous, intrathecal, intra-arterial, intramuscular, intralesional, and subcutaneous administration routes.

Determination of a therapeutically effective amount of the active agent is well within the capability of those skilled in the art, especially in light of the disclosure provided herein.

Administration of the pharmaceutical composition of the invention can be performed once the disease is diagnosed. Administration of the pharmaceutical composition can be performed once a day, every other day, three times a week, twice a week, for a period required to treat or attenuate the disease. Thus, administration can be performed for at least one, two, three, or at least four months, or as long as required to treat the disease and/or prevent the occurrence of symptoms thereof.

The exact formulation, route of administration and dosage can be chosen by the individual physician in view of the patient's condition, for example, weight, age, the severity of the disease (see e.g., Fingl, et al., 1975, in “The Pharmacological Basis of Therapeutics”, Ch. 1 p. 1).

It is to be understood that each possibility mentioned throughout the specification represents a separate embodiment of the invention.

### EXAMPLES

The following abbreviations are used in the examples, description and claims:
LSD: lysosome storage disease
LOTS: Late Onset Tay-Sachs
GM2: ganglioside monosialic 2
Tr: transferrin receptor binding peptide
G-CSF: granulocyte colony stimulating factor
ACE: angiotensin converting enzyme
HEXA: α subunit of β hexosaminidase A
BDNF: Brain derived neurotrophic factor

### Example 1

### Expression of Fusion Proteins Using Different Peptide Linkers

DNA sequences of the fusion proteins were cloned into pTT5 mammalian expression vector and consecutively expressed under the regulation of CMV promoter. The fusion proteins were produced in vitro by the cellular expression system Expi293F™ which secrets the fusion protein to the extracellular medium.

The fusion proteins were concentrated and purified by binding to Ni-column via the C-terminus six-histidine tail and eluted from the Ni-column using an imidazole gradient (5-500 mM). The eluted protein peaks were collected, concentrated, dialyzed in a reconstitution buffer which contained phosphate buffer saline (PBS), 3% mannitol, and 0.01% Tween 80, and then kept at −80° C. Alternatively, the eluted protein peaks were collected, dialyzed in 50 mM ammonium acetate buffer pH 4.5 to remove the imidazole buffer, and the proteins freeze-dried and kept until use at −80° C.

Below are listed the amino acid sequence of the peptide linkers examined:

Angiotensin converting enzyme (ACE) cleavage
site-GS linker (flexible linker;
SEQ ID NO: 16):
GSGSDRVYIHPFHLGSGSGGSGGSGGSGSGGSGSGGSGGSG
Angiotensin converting enzyme (ACE) cleavage
site-GS linker (flexible linker;
SEQ ID NO: 17):
GSGSGSGSGGSGGSDRVYIHPFHLGGSGSGGSGSGGSGGSG
Angiotensin converting enzyme (ACE) cleavage
site-rigid linker (α helical linker;
SEQ ID NO: 22):
AEAAAAKEAAAAKEAAAAKEAAAAKALEAEAAAAKEAAAAKE
AAAAKEAAAAKADRVYIHPFHLG
Angiotensin converting enzyme (ACE) cleavage
site-rigid linker
(α helical linker; SEQ ID NO: 23):
AEAAAAKEAAAAKEAAAAKEAAAAKALEDRVYIHPFHLGAEAA
AAKEAAAAKEAAAAKEAAAAKACCC

In order to select fusion proteins which are more expressible and active, an assay of immunofluorescent staining in cultured human Tay-Sachs glia cells was performed.

Tay-Sachs glia cells (originally derived from a Tay-Sachs embryo, kindly provided by Prof. Ruth Navon) were seeded on 8 wells-multi-chambered slides (purchased from Merck) and incubated for 24 hours in the presence or absence of 80 ng/μl of Tr-G-CSF-HEXA containing different peptide linkers. The cells were then rinsed three times with PBS, fixed for 10 min with 4% Paraformaldehyde at room temperature (RT) followed by 10 min permeabilization with 0.3% TritonX-100 in PBS at RT. Thereafter, the cells were incubated with 5% BSA containing 5% goat serum for 60 min, and then with rabbit anti-GM2 antibodies (purchased from Abcam) for 60 min. Then, the cells were rinsed three times with 0.01% Tween-20 (PBST) followed by incubation with Alexa Fluor™ goat anti-rabbit IgG antibodies (purchased from Invitrogen). The cells were rinsed three times with PBST, mounted with mounting-medium for fluorescence (purchased from Vector Laboratories Incorporation), and observed in confocal microscope using a filter for FITC.

Various peptide linkers were used to link the two-domain carrier protein, mouse Tr-G-CSF, at the C-terminus of G-CSF to the N-terminus of the lysosomal enzyme, mouse HEXA, in order to produce fusion proteins having the different domains organized in the following structure from the N- to the C-terminus: Tr-G-CSF-peptide linker-HEXA.

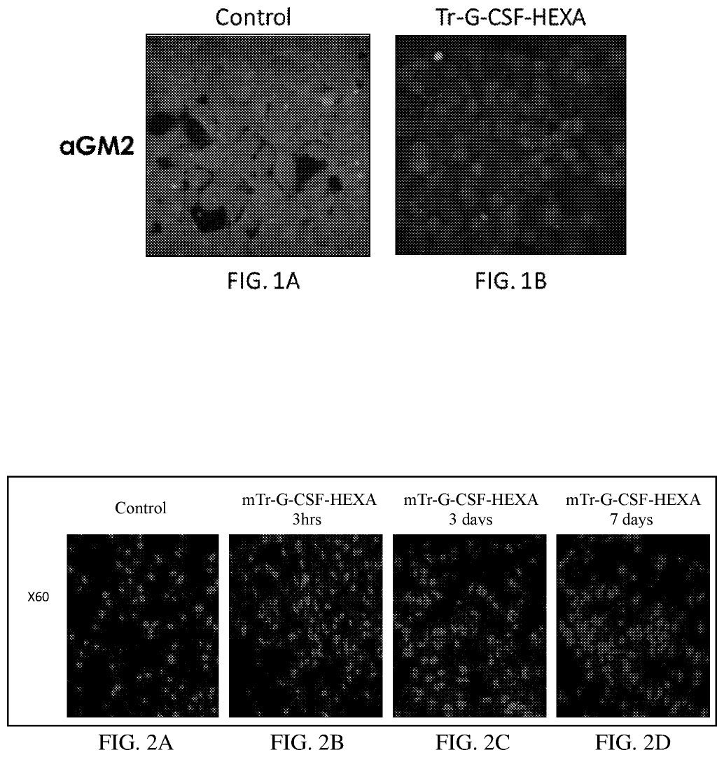

The results indicated that fusion proteins which included a peptide linker having the ACE cleavage site, as set forth in SEQ ID NO:21, and a peptide having a flexible structure attributed to the presence of Gly-Ser repeats, e.g., peptide linkers of SEQ ID NOs:16 and 17; such fusion proteins were successfully expressed and were active in degrading GM2 in Tay-Sachs glia cells in vitro. Representative results are shown in FIGS. 1A-B. FIGS. 1A-B show the GM2 immunofluorescent staining in cultured Tay-Sachs glia cells incubated in the absence (control; FIG. 1A) or in the presence of Tr-G-CSF-HEXA having the peptide linker of SEQ ID NO:16 (FIG. 1B). As shown in FIG. 1B, the fusion protein Tr-G-CSF-HEXA having the peptide linker of SEQ ID NO:16 significantly reduced GM2 content in Tay-Sachs glia cells.

However, fusion proteins which included a peptide linker having the ACE cleavage site and a peptide having a rigid α helical structure attributed to the presence of Glu-Ala-Ala-Ala-Ala-Lys repeats, e.g., peptide linkers of SEQ ID NOs:22 and 23, were inactive in degrading ganglioside GM2 under the same experimental in vitro conditions, as indicated by their failure to lower GM2 immunofluorescent staining in cultured human Tay-Sachs glia cells.

The in vivo studies were therefore performed using mouse fusion proteins comprising Tr-GCSF-peptide linker-HEXA, wherein the peptide linker has a flexible linker, i.e., the peptide linker of SEQ ID NO:16.

### Example 2

### Immunohistochemical Detection of the Fusion Proteins in Brain

The in vivo penetration of the fusion protein mouse Tr-G-CSF-HEXA through the BBB into the brain was tested in wild type mice which received a single intravenous injection of 0.5 mg mouse Tr-G-CSF-HEXA (FIG. 2B-D). As a control, the mice were injected with vehicle only (FIG. 2A). The mice were sacrificed after 3 hours, 3 days or 7 days. Blood was drained and replaced by perfusion with 30 ml PBS and then with 10 ml of 4% paraformaldehyde. The brain was removed and incubated for 18 hours in PBS containing 4% paraformaldehyde followed by incubation in PBS containing 30% sucrose at 4° C. until the brain sink to the bottom of the tube. The brain was then sliced, and brain slices were adhered on a glass slide, placed in citrate buffer and boiled for 10 min. The boiled brain slices were rinsed 3 times with PBS containing 0.05% Triton X-100 (PBST-100), and incubated with rabbit anti-4 His antibody for 3 hours at room temperature. Then, the slices were rinsed 3 times with PBST-100 and incubated with anti-rabbit-IgG-alexa fluor 555 antibody for 1 hour (red staining), and with DAPI for the last one minute (blue staining). The stained-slices were coated with mounting-solution, covered with a cover slip, and visualized by confocal analysis.

As shown in FIG. 2B, Tr-G-CSF-HEXA fusion protein crossed the BBB and was localized in the brain cells three hours after a single injection of the fusion protein. FIGS. 2C-D show that Tr-G-CSF-HEXA was detected in brain cells three days, and to a lesser extent seven days, after Tr-G-CSF-HEXA injection to the mice. No labeling was detected in control mice (FIG. 2A). These results indicate a long-term accumulation of Tr-G-CSF-HEXA fusion protein in the brain.

### Example 3

### Enzymatic Activity of Fusion Proteins in Brain

The in vivo enzymatic activity of the fusion proteins G-CSF-HEXA, Tr-HEXA, and Tr-GCSF-HEXA was then tested.

Tay-Sachs mice^{HEXA-/-} were intravenously received 0.25 mg G-CSF-HEXA of SEQ ID NO:27 twice a week for 7 weeks, 0.25 mg Tr-GCSF-HEXA of SEQ ID NO:29 twice a week for 6 weeks, 0.5 mg Tr-HEXA of SEQ ID NO:28 three times a week for 4 weeks, or vehicle only as a control. Three days after the last administration, the mice were sacrificed and their brains were removed and frozen at −80° C. until GM2 extraction. For GM2 extraction, the brains were homogenized in PBS. GM2 extraction was carried out as follows:
(i) brain homogenate was extracted with Chloroform/Methanol (1:2);
(ii) the lower phase from step (i) was re-extracted with Chloroform/Methanol (2:1);
(iii) the supernatants from steps (i) and (ii) were combined and mixed with water;
(iv) the supernatant/water mixture of step (iii) was extracted with chloroform and the upper phase was collected;
(v) the lower phase of step (iv) was re-extracted with methanol/water (2:1);
(vi) the two upper supernatants of steps (iv) and (v) were combined and dried;
(vii) the dried material was suspended with water and extracted twice with butanol;
(viii) the butanol phases were combined and dried; and
(ix) the dried pellet was dissolved in methanol.
   The amount of GM2 in the methanol solution was measured by LC-MS-MS.

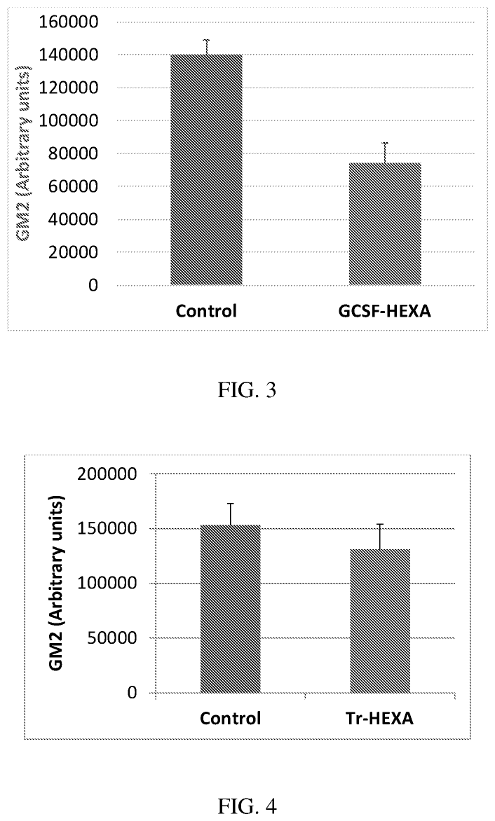

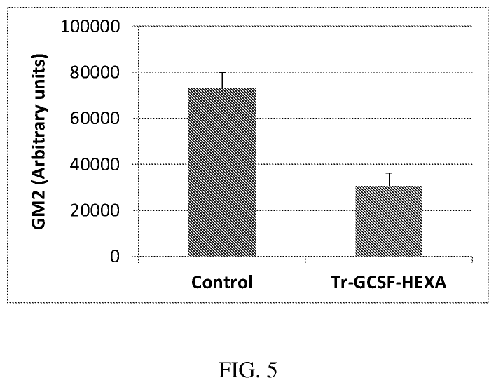

As shown in FIGS. 3-5, all the three fusion proteins reduced the accumulated GM2 amount in the brain of Tay-Sachs mice^{HEXA-/-}. While G-CSF-HEXA (FIG. 3) and Tr-HEXA (FIG. 4) reduced brain GM2 content by 47% and 15%, respectively, Tr-GCSF-HEXA (FIG. 5) reduced the content of brain GM2 by 60%, as compared to the control groups. In these experiments, G-CSF-HEXA was administered at a total amount of 42 nmol, Tr-HEXA was administered at a total amount of 96 nmol, and Tr-GCSF-HEXA was administered at a total amount of 36 nmol. Thus, although Tr-GCSF-HEXA was administered at a lower total amount than G-CSF-HEXA or Tr-HEXA, Tr-GCSF-HEXA was shown to be more effective in degrading GM2 in the brain of Tay-Sachs mice^{HEXA-/-} than each of the other two fusion proteins. Moreover, Tr-GCSF-HEXA was found to show an additive effect under these experimental conditions. Bearing in mind that Tr-GCSF-HEXA was administered at a total amount lower than each of the other fusion proteins, it is assumed that if given at an equal amount as of G-CSF-HEXA, Tr-GCSF-HEXA could have exerted a synergistic effect on GM2 degradation in the brain of Tay-Sachs mice^{HEXA-/-}. Therefore, these results indicate that Tr-GCSF-HEXA is a highly effective means for enzyme replacement therapy in Tay-Sachs patients, and it is significantly more active than G-CSF-HEXA or Tr-HEXA.

### Example 4

### Fusion Proteins of BDNF are Biologically Active

The aim of this study was to produce a fusion protein containing BDNF as an active agent and to evaluate its biological activity.

The following domains were used to form the fusion protein:
(i) a leader sequence (signal peptide) of mouse proBDNF of the amino acid sequence: MTILFLTMVISYFGCMKA (SEQ ID NO:39). A leader sequence (Signal peptide) of mouse Ig Kappa secretory element of the amino acid sequence: ETDTLLLWVLLLWVPGSTGD (SEQ ID NO:25) can also be used;
(ii) mouse proBDNF of SEQ ID NO:32 which includes the mouse pro-domain of BDNF and mouse BDNF was cloned from mouse brain cerebellum. It is noted that while the amino acid sequence of mouse pro-domain of BDNF is different from that of human pro-domain, the sequence of mature BDNF is identical in these two species. In order to produce the mature form of BDNF-GCSF-Tr, the native furin cleavage site of the sequence RVRR (SEQ ID NO:33) was used. This cleavage site can be replaced by the enterokinase cleavage site of the sequence NNNNK (SEQ ID NO:34);
(iii) a Gly-Ser peptide linker of the amino acid sequence GTGSAGSAAGSGEF (SEQ ID NO:35);
(iv) Mouse G-CSF of SEQ ID NO:19;
(v) Transferrin receptor binding peptide containing a Gly-Ser linker at its amino terminus, the amino acid sequence of which is: GSGSGSHAIYPRH (SEQ ID NO:36); and
(vi) Histidine tail.

The resulting fusion protein had the following structure from the N-terminus to the C-terminus: proBDNF-peptide linker-G-CSF-Tr-histidine tail. For brevity, the fusion protein is designated herein below proBDNF-GCSF-Tr.

### Production of proBDNF-GCSF-Tr

The mammalian vector pTT5 was used for cloning. pTT5-proBDNF-G-CSF-Tr (SEQ ID NO:46) was transfected to 293XP cells with ExpiFectamine™ 293 Transfection Kit (purchased from ThermoFisher) and incubated for 3-4 days in a humidified CO₂ incubator under agitation according to the manufacturer instructions. The medium was collected and loaded to HisTrep™ Ni column (purchased from Cytiva) in the presence of 1.5% mannitol, 0.1% TWIN80® (T80) and 10 mM imidazole. Mouse proBDNF-G-CSF-Tr protein (SEQ ID NO:50) was eluted using a 10-500 mM imidazole gradient in phosphate buffer pH 8, and the protein peaks were concentrated and dialyzed against PBS containing 3% mannitol and 0.01% T80, and kept at −20° C. for in vitro experiments.

### Purification of proBDNF-GCSF-Tr

ProBDNF-GCSF-Tr protein was eluted in fractions 12-17 (F12-17) from the Ni column and was identified by Western blot analysis. The C-terminus of the protein was recognized by mouse anti-tetra-HIS antibody (purchased from Qiagen) followed by anti-mouse HRP antibodies (purchased from Jackson). The N-terminal-pro domain was recognized by rabbit-anti-human-BDNF-pro domain (purchased from Alomone labs), and the BDNF domain was recognized by rabbit-anti-human-BDNF (purchased from Alomone labs). Both first antibodies were probed by anti-Rabbit-HRP antibodies (purchased from Jackson).

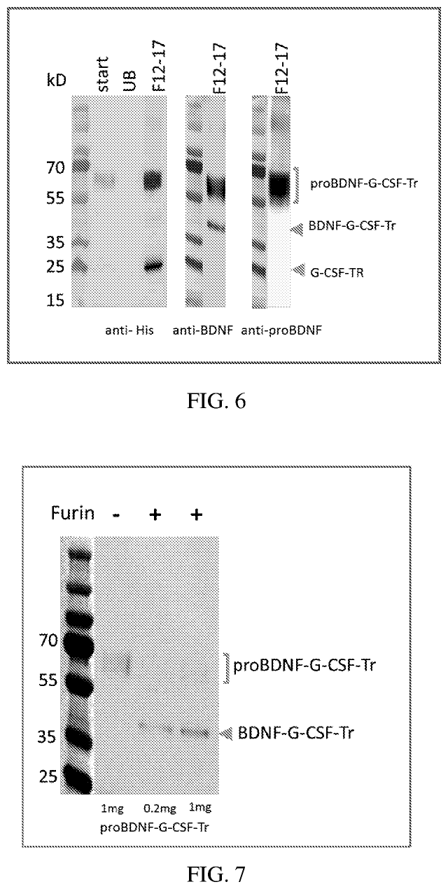

As shown in FIG. 6, the anti-histidine antibodies identified both the proBDNF-GCSF-Tr and GCSF-Tr of apparent MW of 55-70 and 25 kDa, respectively. However, proBDNF-GCSF-Tr appeared to be the major product. When anti-BDNF antibodies were used for immunoblotting, both proBDNF-GCSF-Tr and BDNF-GCSF-Tr were identified (apparent molecular weight of 55-70 and 45 kDa, respectively), however proBDNF-GCSF-Tr appeared to be the major product purified from the Ni column (FIG. 6). The anti-proBDNF antibodies recognized only the proBDNF-GCSF-Tr (FIG. 6). It is noted that the theoretical MW of proBDNF-GCSF-Tr is 50.4 kDa and that of BDNF-GCSF-Tr is 36.9 kDa, suggesting that the differences between the apparent and theoretical MWs is due to increased glycosylation.

Thus, these results indicate that proBDNF-GCSF-Tr was effectively expressed in this mammalian expression system.

### Cleavage of proBDNF-GCSF-Tr

Maturation or cleavage of proBDNF-GCSF-Tr to BDNF-GCSF-Tr can be achieved by enzymatic digestion between the pro-domain and the BDNF domain. While furin can cleave proBDNF at its native cleavage site, i.e., amino acids 127-130 of the sequence RVRR (SEQ ID NO:33), enzymatic digestion using the pancreatic serine protease, enterokinase, by substitution of the amino acids RVRR (Furin recognition and cleavage site) with the amino acid sequences NNNNK (SEQ ID NO:34) at positions 127-131 can also takes place.

The enzymatic cleavage of 1 or 0.2 mg purified proBDNF-GCSF-Tr with 1 Unit of furin was performed in MES buffer pH 7 containing 0.1% TritonX100® (TX100), 1 mM CaCl₂ and 1 mM 2-mercaptoethanol at 30° C. for 4 hours. As a control, 1 mg of proBDNF-GCSF-Tr was incubated at the same conditions in the absence of furin. The digestion products were resolved on SDS PAGE, followed by Western blot with mouse anti-4-His antibodies (purchased from Qiagen) followed by donkey anti-mouse antibody conjugated to Horse Radish Peroxidase (HRP) (purchased from Jackson).

As shown in FIG. 7, furin effectively cleaved the proBDNF-G-CSF-Tr molecule to yield BDNF-G-CSF-Tr.

### Biological Activity of proBDNF-GCSF-Tr

The neurotrophic activity of proBDNF-G-CSF-Tr was demonstrated by its ability to potentiate downstream signaling of its native neurotrophic receptor-Tropomyosin Receptor Kinase B (TRKB). HEK293 cells stably transfected with TRKB (HEKB) were used for this experiment. HEKB cells were grown to 70% confluence, and were serum-depleted for 3 hours. The cells were stimulated with 20 ng/ml BDNF (purchased from Alomone Lab) or with 40 ng/ml proBDNF-G-CSF-Tr for 5 min. TRKB stimulation was assessed by Mitogen Activated Protein Kinase (MAPK) phosphorylation as detected by Western blot analysis with anti-phospho-p42/44 MAPK antibodies (ERK1/2-Tyrosine 204/Threonine 202; purchased from Cell Signaling Technology). As a control, HEKB cells were not stimulated with any protein. In order to confirm that identical amounts of cell proteins were loaded in each lane, Western blot analysis was also performed with rabbit anti AKT (protein kinase B) antibodies (purchased from Cell Signaling Technology) followed by donkey anti-rabbit-antibody conjugated to HRP (purchased from Jackson).

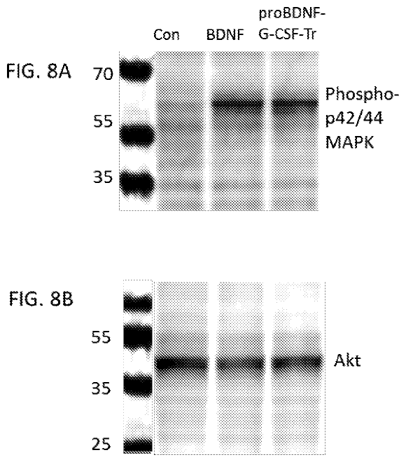

As shown in FIG. 8A, proBDNF-G-CSF-Tr induced phosphorylation of MAPK at the same level as BDNF. Control cells did not show any MAPK phosphorylation. The amount of cell proteins was identical in all groups (FIG. 8B), indicating that the difference in MAPK phosphorylation was not due to different amounts of cell protein. These results indicate that the fusion protein proBDNF-GCSF-Tr was biologically active and retained its neurotrophic activity, i.e., the ability to induce signal transduction, similarly to BDNF.

It will be appreciated by persons skilled in the art that the present invention is not limited by what has been particularly shown and described herein above. Rather the scope of the invention is defined by the claims that follow.

1. A fusion protein comprising:
a two-domain carrier protein and a proteinaceous therapeutic agent linked thereto via a peptide linker,
wherein the two-domain carrier protein comprises a transferrin receptor binding peptide linked to granulocyte colony stimulating factor (G-CSF), and
wherein the fusion protein is capable of passing through the blood brain barrier, thereby transporting the proteinaceous therapeutic agent into, near or onto cells of the central nervous system (CNS) of a subject having a CNS disease.

2.-62. (canceled)

63. The fusion protein according to claim 1, wherein the proteinaceous therapeutic agent is an enzyme, and wherein the fusion protein is capable of replenishing the enzyme in the cells of the CNS at a higher level than a fusion protein comprising the transferrin receptor binding peptide linked to said enzyme and/or at a higher level than a fusion protein comprising the G-CSF linked to the enzyme.

64. The fusion protein according to claim 63, wherein the enzyme is selected from the group consisting of lysosomal enzymes, amyloid beta degrading enzymes, insulin degrading enzyme, and active precursors or fragments thereof.

65. The fusion protein according to claim 64, wherein the lysosomal enzyme is selected from the group consisting of β-hexosaminidase A, aspartylglucosaminidase, acid lipase, α-galactosidase A, acid ceramidase, α-L-fucosidase, α-D-mannosidase, β-D-mannosidase, arylsulphatase A, neuraminidase, α-N-acetylglucosaminidase, phosphotransferase, phosphotransferase γ-subunit, L-iduronidase, iduronate-2-sulphatase, heparan-N-sulphatase, acetylCoA:N-acetyltransferase, N-acetylglucosamine 6-sulphatase, galactose 6-sulphatase, β-galactosidase, N-acetylgalactosamine 4-sulphatase, hyalurono-glucosaminidase, multiple sulphatases, palmitoyl protein thioesterase, tripeptidyl peptidase I, acid sphingomyelinase, cathepsin K, α-galactosidase B, and active precursors or fragments thereof.

66. The fusion protein according to claim 1, wherein the proteinaceous therapeutic agent is a neurotrophic factor.

67. The fusion protein according to claim 66, wherein the neurotrophic factor is selected from the group consisting of brain derived neurotrophic factor (BDNF), nerve growth factor (NGF), glial derived neurotrophic factor (GDNF), neurotrophins (NTs), neurturin, neuregulin, netrin, and ciliary neurotrophic factor (CNTF).

68. The fusion protein according to claim 1, wherein the proteinaceous therapeutic agent is an antibody or an active fragment thereof directed to growth factors or growth factor receptors secreted by or expressed on tumor cells in the brain.

69. The fusion protein according to claim 1, wherein the CNS disease is selected from the group consisting of lysosomal storage diseases, neurodegenerative diseases, and primary or metastatic brain tumors.

70. The fusion protein according to claim 69, wherein the lysosomal enzyme is the α subunit of β-hexosaminidase A comprising the amino acid sequence as set forth in any one of SEQ ID NOs:1-5, or an active fragment thereof.

71. The fusion protein according to claim 1, wherein the transferrin receptor binding peptide comprises the amino acid sequence as set forth in any one of SEQ ID NOs: 6-10, 20, 36, and 37, or an active analog or fragment thereof.

72. The fusion protein according to claim 1, wherein G-CSF comprises the amino acid sequence as set forth in any one SEQ ID NOs:11-15, or an active fragment or analog thereof.

73. The fusion protein according to claim 1, wherein the peptide linker comprises an amino acid sequence having a flexible structure.

74. The fusion protein according to claim 73, wherein the peptide linker comprises a peptide selected from the group consisting of: an angiotensin converting enzyme (ACE) cleavage site, a (Gly-Ser)m peptide wherein m ranges from 1 to 30, a (Gly-Gly-Ser)n peptide wherein n ranges from 1 to 20, a peptide having the sequence of GTGSAGSAAGSGEF (SEQ ID NO:35), an analog, fragment and combinations thereof.

75. The fusion protein according to claim 1,
wherein the lysosomal enzyme is the α subunit of β-hexosaminidase A comprising the amino acid sequence as set forth in any one of SEQ ID NOs:1-5, or an active fragment thereof,
wherein the transferrin receptor binding peptide comprises the amino acid sequence as set forth in any one of SEQ ID NOs:6-10, 20, 36 and 37, or an active analog or fragment thereof, and
wherein G-CSF comprises the amino acid sequence as set forth in any one SEQ ID NOs:11-15, or an active fragment or analog thereof.

76. The fusion protein according to claim 75,
wherein the neurotrophic factor is BDNF or a precursor thereof comprising the amino acid sequence as set forth in any one of SEQ ID NOs: 30 and 31, or an active fragment or analog thereof,
wherein G-CSF comprises the amino acid sequence as set forth in any one of SEQ ID NOs:11-15, or an active fragment or analog thereof, and
wherein the transferrin receptor binding peptide comprises the amino acid sequence as set forth in any one of SEQ ID NOs:36 and 37, or an active analog or fragment thereof.

77. A polynucleotide encoding the fusion protein according to claim 1.

78. An expression vector comprising the polynucleotide according to claim 77.

79. A host cell comprising the expression vector according to claim 78.

80. A pharmaceutical composition comprising as an active agent at least one of the following agents: (i) a fusion protein according to claim 1; (ii) a polynucleotide encoding the fusion protein of (i); (iii) an expression vector comprising the polynucleotide of (ii); and (iv) a host cell comprising the expression vector of (iii); and a pharmaceutically acceptable carrier.

81. A method of treating a CNS disease comprising administering to a subject in need of such treatment a therapeutically effective amount of a pharmaceutical composition according to claim 80, thereby treating the CNS disease.


SEQUENCE LISTING (51 sequences)

SEQ ID NO: 1 - protein, 507 aa, Artificial Sequence
  Synthetic peptide
LWPWPQNFQTSDQRYVLYPNNFQFQYDVSSAAQPGCSVLDEAFQRYRDLLFGSGSWPRPYLTGKRHTLEKNVLVVSVVTPGCNQLPTLESVENYTLTINDDQCLLLSETVWGALRGLETFSQLVWKSAEGTFFINKTEIEDFPRFPHRGLLLDTSRHYLPLSSILDTLDVMAYNKLNVFHWHLVDDPSFPYESFTFPELMRKGSYNPVTHIYTAQDVKEVIEYARLRGIRVLAEFDTPGHTLSWGPGIPGLLTPCYSGSEPSGTFGPVNPSLNNTYEFMSTFFLEVSSVFPDFYLHLGGDEVDFTCWKSNPEIQDFMRKKGFGEDFKQLESFYIQTLLDIVSSYGKGYVVWQEVFDNKVKIQPDTIIQVWREDIPVNYMKELELVTKAGFRALLSAPWYLNRISYGPDWKDFYIVEPLAFEGTPEQKALVIGGEACMWGEYVDNTNLVPRLWPRAGAVAERLWSNKLTSDLTFAYERLSHFRCELLRRGVQAQPLNVGFCEQEFEQT

SEQ ID NO: 2 - protein, 529 aa, Artificial Sequence
  Synthetic peptide
MTSSRLWFSLLLAAAFAGRATALWPWPQNFQTSDQRYVLYPNNFQFQYDVSSAAQPGCSVLDEAFQRYRDLLFGSGSWPRPYLTGKRHTLEKNVLVVSVVTPGCNQLPTLESVENYTLTINDDQCLLLSETVWGALRGLETFSQLVWKSAEGTFFINKTEIEDFPRFPHRGLLLDTSRHYLPLSSILDTLDVMAYNKLNVFHWHLVDDPSFPYESFTFPELMRKGSYNPVTHIYTAQDVKEVIEYARLRGIRVLAEFDTPGHTLSWGPGIPGLLTPCYSGSEPSGTFGPVNPSLNNTYEFMSTFFLEVSSVFPDFYLHLGGDEVDFTCWKSNPEIQDFMRKKGFGEDFKQLESFYIQTLLDIVSSYGKGYVVWQEVFDNKVKIQPDTIIQVWREDIPVNYMKELELVTKAGFRALLSAPWYLNRISYGPDWKDFYIVEPLAFEGTPEQKALVIGGEACMWGEYVDNTNLVPRLWPRAGAVAERLWSNKLTSDLTFAYERLSHFRCELLRRGVQAQPLNVGFCEQEFEQT

SEQ ID NO: 3 - protein, 441 aa, Artificial Sequence
  Synthetic peptide
TLEKNVLVVSVVTPGCNQLPTLESVENYTLTINDDQCLLLSETVWGALRGLETFSQLVWKSAEGTFFINKTEIEDFPRFPHRGLLLDTSRHYLPLSSILDTLDVMAYNKLNVFHWHLVDDPSFPYESFTFPELMRKGSYNPVTHIYTAQDVKEVIEYARLRGIRVLAEFDTPGHTLSWGPGIPGLLTPCYSGSEPSGTFGPVNPSLNNTYEFMSTFFLEVSSVFPDFYLHLGGDEVDFTCWKSNPEIQDFMRKKGFGEDFKQLESFYIQTLLDIVSSYGKGYVVWQEVFDNKVKIQPDTIIQVWREDIPVNYMKELELVTKAGFRALLSAPWYLNRISYGPDWKDFYIVEPLAFEGTPEQKALVIGGEACMWGEYVDNTNLVPRLWPRAGAVAERLWSNKLTSDLTFAYERLSHFRCELLRRGVQAQPLNVGFCEQEFEQT

SEQ ID NO: 4 - protein, 191 aa, Artificial Sequence
  Synthetic peptide
MTSSRLWFSLLLAAAFAGRATALWPWPQNFQTSDQRYVLYPNNFQFQYDVSSAAQPGCSVLDEAFQRYRDLLFGSGSWPRPYLTGKRHTLEKNVLVVSVVTPGCNQLPTLESVENYTLTINDDQCLLLSETVWGALRGLETFSQLVWKSAEGTFFINKTEIEDFPRFPHRGLLLDTSRHYLPLSSILDTLD

SEQ ID NO: 5 - protein, 127 aa, Artificial Sequence
  Synthetic peptide
ELELVTKAGFRALLSAPWYLNRISYGPDWKDFYIVEPLAFEGTPEQKALVIGGEACMWGEYVDNTNLVPRLWPRAGAVAERLWSNKLTSDLTFAYERLSHFRCELLRRGVQAQPLNVGFCEQEFEQT

SEQ ID NO: 6 - protein, 11 aa, Artificial Sequence
  Synthetic peptide
HAIYPRHGSSR

SEQ ID NO: 7 - protein, 12 aa, Artificial Sequence
  Synthetic peptide
THRPPMWSPVWP

SEQ ID NO: 8 - protein, 51 aa, Artificial Sequence
  Synthetic peptide
GSREGCASRCTKYNAELEKCEARVSSMSNTEETCVQELFDLLHCVDHCVSQ

SEQ ID NO: 9 - protein, 51 aa, Artificial Sequence
  Synthetic peptide
GSREGCASRCTKYNAELEKCEARVMSMSNTEEDCEQELEDLLHCLDHCHSQ

SEQ ID NO: 10 - protein, 51 aa, Artificial Sequence
  Synthetic peptide
GSREGCASRCMKYNDELEKCEARMMSMSNTEEDCEQELEDLLYCLDHCHSQ

SEQ ID NO: 11 - protein, 175 aa, Artificial Sequence
  Synthetic peptide
ATPLGPASSLPQSFLLKCLEQVRKIQGDGAALQEKLCATYKLCHPEELVLLGHSLGIPWAPLSSCPSQALQLAGCLSQLHSGLFLYQGLLQALEGISPELGPTLDTLQLDVADFATTIWQQMEELGMAPALQPTQGAMPAFASAFQRRAGGVLVASHLQSFLEVSYRVLRHLAQP

SEQ ID NO: 12 - protein, 207 aa, Artificial Sequence
  Synthetic peptide
MAGPATQSPMKLMALQLLLWHSALWTVQEATPLGPASSLPQSFLLKCLEQVRKIQGDGAALQEKLVSECATYKLCHPEELVLLGHSLGIPWAPLSSCPSQALQLAGCLSQLHSGLFLYQGLLQALEGISPELGPTLDTLQLDVADFATTIWQQMEELGMAPALQPTQGAMPAFASAFQRRAGGVLVASHLQSFLEVSYRVLRHLAQP

SEQ ID NO: 13 - protein, 177 aa, Artificial Sequence
  Synthetic peptide
TPLGPASSLPQSFLLKCLEQVRKIQGDGAALQEKLVSECATYKLCHPEELVLLGHSLGIPWAPLSSCPSQALQLAGCLSQLHSGLFLYQGLLQALEGISPELGPTLDTLQLDVADFATTIWQQMEELGMAPALQPTQGAMPAFASAFQRRAGGVLVASHLQSFLEVSYRVLRHLAQP

SEQ ID NO: 14 - protein, 204 aa, Artificial Sequence
  Synthetic peptide
MAGPATQSPMKLMALQLLLWHSALWTVQEATPLGPASSLPQSFLLKCLEQVRKIQGDGAALQEKLVSECATYKLCHPEELVLLGHSLGIPWAPLSSCPSQALQLAGCLSQLHSGLFLYQGLLQALEGISPELGPTLDTLQLDVADFATTIWQQMEELGMAPALQPTQGAMPAFASAFQRRAGGVLVASHLQSFLEVSYRVLRHL

SEQ ID NO: 15 - protein, 174 aa, Artificial Sequence
  Synthetic peptide
TPLGPASSLPQSFLLKCLEQVRKIQGDGAALQEKLVSECATYKLCHPEELVLLGHSLGIPWAPLSSCPSQALQLAGCLSQLHSGLFLYQGLLQALEGISPELGPTLDTLQLDVADFATTIWQQMEELGMAPALQPTQGAMPAFASAFQRRAGGVLVASHLQSFLEVSYRVLRHL

SEQ ID NO: 16 - protein, 41 aa, Artificial Sequence
  Synthetic peptide
GSGSDRVYIHPFHLGSGSGGSGGSGGSGSGGSGSGGSGGSG

SEQ ID NO: 17 - protein, 41 aa, Artificial Sequence
  Synthetic peptide
GSGSGSGSGGSGGSDRVYIHPFHLGGSGSGGSGSGGSGGSG

SEQ ID NO: 18 - protein, 764 aa, Artificial Sequence
  Synthetic peptide
MGETDTLLLWVLLLWVPGSTGDHAIYPRHGSSRGSGSATPLGPASSLPQSFLLKCLEQVRKIQGDGAALQEKLCATYKLCHPEELVLLGHSLGIPWAPLSSCPSQALQLAGCLSQLHSGLFLYQGLLQALEGISPELGPTLDTLQLDVADFATTIWQQMEELGMAPALQPTQGAMPAFASAFQRRAGGVLVASHLQSFLEVSYRVLRHLAQPGSGSGSGSGSGGSGGSDRVYIHPFHLGGSGSGGSGSGGSGGSGELLWPWPQNFQTSDQRYVLYPNNFQFQYDVSSAAQPGCSVLDEAFQRYRDLLFGSGSWPRPYLTGKRHTLEKNVLVVSVVTPGCNQLPTLESVENYTLTINDDQCLLLSETVWGALRGLETFSQLVWKSAEGTFFINKTEIEDFPRFPHRGLLLDTSRHYLPLSSILDTLDVMAYNKLNVFHWHLVDDPSFPYESFTFPELMRKGSYNPVTHIYTAQDVKEVIEYARLRGIRVLAEFDTPGHTLSWGPGIPGLLTPCYSGSEPSGTFGPVNPSLNNTYEFMSTFFLEVSSVFPDFYLHLGGDEVDFTCWKSNPEIQDFMRKKGFGEDFKQLESFYIQTLLDIVSSYGKGYVVWQEVFDNKVKIQPDTIIQVWREDIPVNYMKELELVTKAGFRALLSAPWYLNRISYGPDWKDFYIVEPLAFEGTPEQKALVIGGEACMWGEYVDNTNLVPRLWPRAGAVAERLWSNKLTSDLTFAYERLSHFRCELLRRGVQAQPLNVGFCEQEFEQT

SEQ ID NO: 19 - protein, 178 aa, Artificial Sequence
  Synthetic peptide
VPLVTVSALPPSLPLPRSFLLKSLEQVRKIQASGSVLLEQLCATYKLCHPEELVLLGHSLGIPKASLSGCSSQALQQTQCLSQLHSGLCLYQGLLQALSGISPALAPTLDLLQLDVANFATTIWQQMENLGVAPTVQPTQSAMPAFTSAFQRRAGGVLAISYLQGFLETARLALHHLA

SEQ ID NO: 20 - protein, 9 aa, Artificial Sequence
  Synthetic peptide
HAIYPRHGS

SEQ ID NO: 21 - protein, 10 aa, Artificial Sequence
  Synthetic peptide
DRVYIHPFHL

SEQ ID NO: 22 - protein, 65 aa, Artificial Sequence
  Synthetic peptide
AEAAAAKEAAAAKEAAAAKEAAAAKALEAEAAAAKEAAAAKEAAAAKEAAAAKADRVYIHPFHLG

SEQ ID NO: 23 - protein, 68 aa, Artificial Sequence
  Synthetic peptide
AEAAAAKEAAAAKEAAAAKEAAAAKALEDRVYIHPFHLGAEAAAAKEAAAAKEAAAAKEAAAAKACCC

SEQ ID NO: 24 - protein, 506 aa, Artificial Sequence
  Synthetic peptide
LWPWPQYIQTYHRRYTLYPNNFQFRYHVSSAAQAGCVVLDEAFRRYRNLLFGSGSWPRPSFSNKQQTLGKNILVVSVVTAECNEFPNLESVENYTLTINDDQCLLASETVWGALRGLETFSQLVWKSAEGTFFINKTKIKDFPRFPHRGVLLDTSRHYLPLSSILDTLDVMAYNKFNVFHWHLVDDSSFPYESFTFPELTRKGSFNPVTHIYTAQDVKEVIEYARLRGIRVLAEFDTPGHTLSWGPGAPGLLTPCYSGSHLSGTFGPVNPSLNSTYDFMSTLFLEISSVFPDFYLHLGGDEVDFTCWKSNPNIQAFMKKKGFTDFKQLESFYIQTLLDIVSDYDKGYVVWQEVFDNKVKVRPDTIIQVWREEMPVEYMLEMQDITRAGFRALLSAPWYLNRVKYGPDWKDMYKVEPLAFHGTPEQKALVIGGEACMWGEYVDSTNLVPRLWPRAGAVAERLWSSNLTTNIDFAFKRLSHFRCELVRRGIQAQPISVGYCEQEFEQT

SEQ ID NO: 25 - protein, 20 aa, Artificial Sequence
  Synthetic peptide
ETDTLLLWVLLLWVPGSTGD

SEQ ID NO: 26 - protein, 15 aa, Artificial Sequence
  Synthetic peptide
GTENLYFQGHHHHHH

SEQ ID NO: 27 - protein, 750 aa, Artificial Sequence
  Synthetic peptide
METDTLLLWVLLLWVPGSTGDVPLVTVSALPPSLPLPRSFLLKSLEQVRKIQASGSVLLEQLCATYKLCHPEELVLLGHSLGIPKASLSGCSSQALQQTQCLSQLHSGLCLYQGLLQALSGISPALAPTLDLLQLDVANFATTIWQQMENLGVAPTVQPTQSAMPAFTSAFQRRAGGVLAISYLQGFLETARLALHHLAELGSGSDRVYIHPFHLGSGSGGSGGSGGSGSGGSGSGGSGGSGSRLWPWPQYIQTYHRRYTLYPNNFQFRYHVSSAAQAGCVVLDEAFRRYRNLLFGSGSWPRPSFSNKQQTLGKNILVVSVVTAECNEFPNLESVENYTLTINDDQCLLASETVWGALRGLETFSQLVWKSAEGTFFINKTKIKDFPRFPHRGVLLDTSRHYLPLSSILDTLDVMAYNKFNVFHWHLVDDSSFPYESFTFPELTRKGSFNPVTHIYTAQDVKEVIEYARLRGIRVLAEFDTPGHTLSWGPGAPGLLTPCYSGSHLSGTFGPVNPSLNSTYDFMSTLFLEISSVFPDFYLHLGGDEVDFTCWKSNPNIQAFMKKKGFTDFKQLESFYIQTLLDIVSDYDKGYVVWQEVFDNKVKVRPDTIIQVWREEMPVEYMLEMQDITRAGFRALLSAPWYLNRVKYGPDWKDMYKVEPLAFHGTPEQKALVIGGEACMWGEYVDSTNLVPRLWPRAGAVAERLWSSNLTTNIDFAFKRLSHFRCELVRRGIQAQPISVGYCEQEFEQT

SEQ ID NO: 28 - protein, 539 aa, Artificial Sequence
  Synthetic peptide
MGETDTLLLWVLLLWVPGSTGDHAIYPRHGSSRLWPWPQYIQTYHRRYTLYPNNFQFRYHVSSAAQAGCVVLDEAFRRYRNLLFGSGSWPRPSFSNKQQTLGKNILVVSVVTAECNEFPNLESVENYTLTINDDQCLLASETVWGALRGLETFSQLVWKSAEGTFFINKTKIKDFPRFPHRGVLLDTSRHYLPLSSILDTLDVMAYNKFNVFHWHLVDDSSFPYESFTFPELTRKGSFNPVTHIYTAQDVKEVIEYARLRGIRVLAEFDTPGHTLSWGPGAPGLLTPCYSGSHLSGTFGPVNPSLNSTYDFMSTLFLEISSVFPDFYLHLGGDEVDFTCWKSNPNIQAFMKKKGFTDFKQLESFYIQTLLDIVSDYDKGYVVWQEVFDNKVKVRPDTIIQVWREEMPVEYMLEMQDITRAGFRALLSAPWYLNRVKYGPDWKDMYKVEPLAFHGTPEQKALVIGGEACMWGEYVDSTNLVPRLWPRAGAVAERLWSSNLTTNIDFAFKRLSHFRCELVRRGIQAQPISVGYCEQEFEQT

SEQ ID NO: 29 - protein, 764 aa, Artificial Sequence
  Synthetic peptide
MGETDTLLLWVLLLWVPGSTGDHAIYPRHGSGSGSVPLVTVSALPPSLPLPRSFLLKSLEQVRKIQASGSVLLEQLCATYKLCHPEELVLLGHSLGIPKASLSGCSSQALQQTQCLSQLHSGLCLYQGLLQALSGISPALAPTLDLLQLDVANFATTIWQQMENLGVAPTVQPTQSAMPAFTSAFQRRAGGVLAISYLQGFLETARLALHHLAELGSGSDRVYIHPFHLGSGSGGSGGSGGSGSGGSGSGGSGGSGSRLWPWPQYIQTYHRRYTLYPNNFQFRYHVSSAAQAGCVVLDEAFRRYRNLLFGSGSWPRPSFSNKQQTLGKNILVVSVVTAECNEFPNLESVENYTLTINDDQCLLASETVWGALRGLETFSQLVWKSAEGTFFINKTKIKDFPRFPHRGVLLDTSRHYLPLSSILDTLDVMAYNKFNVFHWHLVDDSSFPYESFTFPELTRKGSFNPVTHIYTAQDVKEVIEYARLRGIRVLAEFDTPGHTLSWGPGAPGLLTPCYSGSHLSGTFGPVNPSLNSTYDFMSTLFLEISSVFPDFYLHLGGDEVDFTCWKSNPNIQAFMKKKGFTDFKQLESFYIQTLLDIVSDYDKGYVVWQEVFDNKVKVRPDTIIQVWREEMPVEYMLEMQDITRAGFRALLSAPWYLNRVKYGPDWKDMYKVEPLAFHGTPEQKALVIGGEACMWGEYVDSTNLVPRLWPRAGAVAERLWSSNLTTNIDFAFKRLSHFRCELVRRGIQAQPISVGYCEQEFEQT

SEQ ID NO: 30 - protein, 119 aa, Artificial Sequence
  Synthetic peptide
HSDPARRGELSVCDSISEWVTAADKKTAVDMSGGTVTVLEKVPVSKGQLKQYFYETKCNPMGYTKEGCRGIDKRHWNSQCRTTQSYVRALTMDSKKRIGWRFIRIDTSCVCTLTIKRGR

SEQ ID NO: 31 - protein, 231 aa, Artificial Sequence
  Synthetic peptide
APMKEVNVHGQGNLAYPGVRTHGTLESVNGPRAGSRGLTTTSLADTFEHVIEELLDEDQKVRPNEENHKDADLYTSRVMLSSQVPLEPPLLFLLEEYKNYLDAANMSMRVRRHSDPARRGELSVCDSISEWVTAADKKTAVDMSGGTVTVLEKVPVSKGQLKQYFYETKCNPMGYTKEGCRGIDKRHWNSQCRTTQSYVRALTMDSKKRIGWRFIRIDTSCVCTLTIKRGR

SEQ ID NO: 32 - protein, 229 aa, Artificial Sequence
  Synthetic peptide
APMKEANIRGQGGLAYPGVRTHGTLESVNGPKAGSRGLTSLADTFEHVIEELLDEDQKVRPNEENNKDADLYTSRVMLSSQVPLEPPLLFLLEEYKNYLDAANMSMRVRRHSDPARRGELSVCDSISEWVTAADKKTAVDMSGGTVTVLEKVPVSKGQLKQYFYETKCNPMGYTKEGCRGIDKRHWNSQCRTTQSYVRALTMDSKKRIGWRFIRIDTSCVCTLTIKRGR

SEQ ID NO: 33 - protein, 4 aa, Artificial Sequence
  Synthetic peptide
RVRR

SEQ ID NO: 34 - protein, 5 aa, Artificial Sequence
  Synthetic peptide
NNNNK

SEQ ID NO: 35 - protein, 14 aa, Artificial Sequence
  Synthetic peptide
GTGSAGSAAGSGEF

SEQ ID NO: 36 - protein, 13 aa, Artificial Sequence
  Synthetic peptide
GSGSGSHAIYPRH

SEQ ID NO: 37 - protein, 7 aa, Artificial Sequence
  Synthetic peptide
HAIYPRH

SEQ ID NO: 38 - protein, 327 aa, Artificial Sequence
  Synthetic peptide
HSDPARRGELSVCDSISEWVTAADKKTAVDMSGGTVTVLEKVPVSKGQLKQYFYETKCNPMGYTKEGCRGIDKRHWNSQCRTTQSYVRALTMDSKKRIGWRFIRIDTSCVCTLTIKRGRGTGSAGSAAGSGEFATPLGPASSLPQSFLLKCLEQVRKIQGDGAALQEKLCATYKLCHPEELVLLGHSLGIPWAPLSSCPSQALQLAGCLSQLHSGLFLYQGLLQALEGISPELGPTLDTLQLDVADFATTIWQQMEELGMAPALQPTQGAMPAFASAFQRRAGGVLVASHLQSFLEVSYRVLRHLAQPGSGSHAIYPRHGSHHHHHH

SEQ ID NO: 39 - protein, 18 aa, Artificial Sequence
  Synthetic peptide
MTILFLTMVISYFGCMKA

SEQ ID NO: 40 - DNA, 2295 bp, Artificial Sequence
  Synthetic peptide
atgggggagaccgacaccctgctgctgtgggtgctgctgctgtgggtgcccggcagcaccggcgacgccacccctctgggccctgcctccagcctgccccagtccttcctgctgaagtgcctggaacaggtgcgcaagatccagggcgacggcgctgccctgcaggaaaagctctgcgccacctacaagctgtgccaccccgaggaactggtgctgctgggacacagcctgggaatcccttgggcccctctgtcctcctgccctagccaggctctgcagctggccggatgcctgtcccagctgcactccggcctgttcctctaccagggactgctgcaggctctcgaaggcatctcccccgagctgggccctaccctggacacactgcagctggacgtggccgacttcgccaccaccatctggcagcagatggaagaactgggcatggccccagctctgcagcctacccagggcgccatgcctgccttcgcctccgccttccagagaagggctggaggagtgctggtggcctcccacctgcagtcattcctcgaagtgtcctaccgcgtgctgaggcacctggcccagcccggatccggcagcggcagcgacagggtgtacatccacccctttcacctgggcagcggaagcggaggaagcggaggaagcggaggaagcggcagcggaggaagcggaagcggaggaagcggaggcagcggctctagactgtggccttggccccagaacttccagacctccgaccagcgctacgtgctgtaccccaacaacttccagttccagtacgacgtgtcctccgccgctcagcctggatgctccgtgctggacgaggccttccagcgctacagggacctgctgttcggctccggctcctggcccaggccctacctgaccggcaagcgccacaccctggaaaagaacgtgctggtggtgtccgtggtgacccccggctgcaaccagctgcccaccctggaatccgtggagaactacaccctgaccatcaacgacgatcagtgcctgctgctgtccgagactgtgtggggagccctgcgcggcctggaaaccttctcccagctggtgtggaagtccgccgagggcaccttcttcatcaacaagaccgagatcgaggacttcccccgcttccctcaccgcggactgctgctggacacctcccgccactacctgcccctgtcctccatcctggacaccctggacgtgatggcctacaacaagctgaacgtgttccactggcacctggtggacgaccccagcttcccctacgagtccttcaccttccccgagctgatgcgcaagggctcttacaaccccgtgacccacatctacaccgcccaggacgtgaaggaagtgatcgagtacgcccgcctgaggggcatcagggtgctggccgagttcgacacccccggacacaccctgtcttggggacccggcatccccggtctgctgaccccctgctactccggctctgagccctccggcaccttcggccccgtgaacccctccctgaacaacacctacgagtttatgtctacattcttcctggaagtgtcatccgtgttccccgacttctacctgcacctgggcggcgacgaggtggacttcacttgctggaagtccaaccccgaaatccaggacttcatgcgcaagaagggcttcggcgaggacttcaagcagctggaatcattctacatccagaccctgctggacatcgtgtcctcttacggcaagggctacgtggtgtggcaggaagtgttcgacaacaaggtgaagatccagcccgacaccatcatccaggtgtggcgcgaggacatccccgtgaactacatgaaggaactggaactggtgaccaaggccggcttcagagccctgctgtccgccccctggtatctgaaccgcatctcctacggccccgactggaaggacttctacatcgtggagcccctggccttcgagggcacccccgagcagaaggccctggtcatcggcggcgaggcctgcatgtggggcgagtacgtggacaacaccaacctggtgcccaggctgtggcctagggctggcgccgtggccgagcgcctgtggtctaacaagctgacctctgacctgaccttcgcctacgagaggctgtcccacttccgctgcgagctgctgagaaggggcgtgcaggcccagcccctgaacgtgggcttctgcgagcaggagttcgagcagactggtaccgagaacctgtacttccaaggccaccaccatcaccatcactaa

SEQ ID NO: 41 - DNA, 2277 bp, Artificial Sequence
  Synthetic peptide
atggagaccgacaccctgctgctgtgggtgctgctgctgtgggtgcccggcagcaccggcgacgtgcccctggtgaccgtgagcgccctgccccccagcctgcccctgcccaggagcttcctgctgaagagcctggagcaggtgaggaagatccaggccagcggcagcgtgctgctggagcagctgtgcgccacctacaagctgtgccaccccgaggagctggtgctgctgggccacagcctgggcatccccaaggccagcctgagcggctgcagcagccaggccctgcagcagacccagtgcctgagccagctgcacagcggcctgtgcctgtaccagggcctgctgcaggccctgagcggcatcagccccgccctggcccccaccctggacctgctgcagctggacgtggccaacttcgccaccaccatctggcagcagatggagaacctgggcgtggcccccaccgtgcagcccacccagagcgccatgcccgccttcaccagcgccttccagaggagggccggcggcgtgctggccatcagctacctgcagggcttcctggagaccgccaggctggccctgcaccacctggccgagctcggcagcggcagcgacagggtgtacatccacccctttcacctgggcagcggaagcggaggaagcggaggaagcggaggaagcggcagcggaggaagcggaagcggaggaagcggaggcagcggctctagactgtggccctggccccagtacatccagacctaccacaggaggtacaccctgtaccccaacaacttccagttcaggtaccacgtgagcagcgccgcccaggccggctgcgtggtgctggacgaggccttcaggaggtacaggaacctgctgttcggcagcggcagctggcccaggcccagcttcagcaacaagcagcagaccctgggcaagaacatcctggtggtgagcgtggtgaccgccgagtgcaacgagttccccaacctggagagcgtggagaactacaccctgaccatcaacgacgaccagtgcctgctggccagcgagaccgtgtggggcgccctgaggggcctggagaccttcagccagctggtgtggaagagcgccgagggcaccttcttcatcaacaagaccaagatcaaggacttccccaggttcccccacaggggcgtgctgctggacaccagcaggcactacctgcccctgagcagcatcctggacaccctggacgtgatggcctacaacaagttcaacgtgttccactggcacctggtggacgacagcagcttcccctacgagagcttcaccttccccgagctgaccaggaagggcagcttcaaccccgtgacccacatctacaccgcccaggacgtgaaggaggtgatcgagtacgccaggctgaggggcatcagggtgctggccgagttcgacacccccggccacaccctgagctggggccccggcgcccccggcctgctgaccccctgctacagcggcagccacctgagcggcaccttcggccccgtgaaccccagcctgaacagcacctacgacttcatgagcaccctgttcctggagatcagcagcgtgttccccgacttctacctgcacctgggcggcgacgaggtggacttcacctgctggaagagcaaccccaacatccaggccttcatgaagaagaagggcttcaccgacttcaagcagctggagagcttctacatccagaccctgctggacatcgtgagcgactacgacaagggctacgtggtgtggcaggaggtgttcgacaacaaggtgaaggtgaggcccgacaccatcatccaggtgtggagggaggagatgcccgtggagtacatgctggagatgcaggacatcaccagggccggcttcagggccctgctgagcgccccctggtacctgaacagggtgaagtacggccccgactggaaggacatgtacaaggtggagcccctggccttccacggcacccccgagcagaaggccctggtgatcggcggcgaggcctgcatgtggggcgagtacgtggacagcaccaacctggtgcccaggctgtggcccagggccggcgccgtggccgagaggctgtggagcagcaacctgaccaccaacatcgacttcgccttcaagaggctgagccacttcaggtgcgagctggtgaggaggggcatccaggcccagcccatcagcgtgggctactgcgagcaggagttcgagcagaccggtacccaccaccatcaccatcactaa

SEQ ID NO: 42 - DNA, 1644 bp, Artificial Sequence
  Synthetic peptide
atgggggagaccgacaccctgctgctgtgggtgctgctgctgtgggtgcccggcagcaccggcgaccacgccatctaccccaggcacggcagctctagactgtggccctggccccagtacatccagacctaccacaggaggtacaccctgtaccccaacaacttccagttcaggtaccacgtgagcagcgccgcccaggccggctgcgtggtgctggacgaggccttcaggaggtacaggaacctgctgttcggcagcggcagctggcccaggcccagcttcagcaacaagcagcagaccctgggcaagaacatcctggtggtgagcgtggtgaccgccgagtgcaacgagttccccaacctggagagcgtggagaactacaccctgaccatcaacgacgaccagtgcctgctggccagcgagaccgtgtggggcgccctgaggggcctggagaccttcagccagctggtgtggaagagcgccgagggcaccttcttcatcaacaagaccaagatcaaggacttccccaggttcccccacaggggcgtgctgctggacaccagcaggcactacctgcccctgagcagcatcctggacaccctggacgtgatggcctacaacaagttcaacgtgttccactggcacctggtggacgacagcagcttcccctacgagagcttcaccttccccgagctgaccaggaagggcagcttcaaccccgtgacccacatctacaccgcccaggacgtgaaggaggtgatcgagtacgccaggctgaggggcatcagggtgctggccgagttcgacacccccggccacaccctgagctggggccccggcgcccccggcctgctgaccccctgctacagcggcagccacctgagcggcaccttcggccccgtgaaccccagcctgaacagcacctacgacttcatgagcaccctgttcctggagatcagcagcgtgttccccgacttctacctgcacctgggcggcgacgaggtggacttcacctgctggaagagcaaccccaacatccaggccttcatgaagaagaagggcttcaccgacttcaagcagctggagagcttctacatccagaccctgctggacatcgtgagcgactacgacaagggctacgtggtgtggcaggaggtgttcgacaacaaggtgaaggtgaggcccgacaccatcatccaggtgtggagggaggagatgcccgtggagtacatgctggagatgcaggacatcaccagggccggcttcagggccctgctgagcgccccctggtacctgaacagggtgaagtacggccccgactggaaggacatgtacaaggtggagcccctggccttccacggcacccccgagcagaaggccctggtgatcggcggcgaggcctgcatgtggggcgagtacgtggacagcaccaacctggtgcccaggctgtggcccagggccggcgccgtggccgagaggctgtggagcagcaacctgaccaccaacatcgacttcgccttcaagaggctgagccacttcaggtgcgagctggtgaggaggggcatccaggcccagcccatcagcgtgggctactgcgagcaggagttcgagcagaccggtacccaccaccatcaccatcactaa

SEQ ID NO: 43 - DNA, 2319 bp, Artificial Sequence
  Synthetic peptide
atgggggagaccgacaccctgctgctgtgggtgctgctgctgtgggtgcccggcagcaccggcgaccacgccatctaccccaggcacggcagcggaagcggcagcgtgcccctggtgaccgtgagcgccctgccccccagcctgcccctgcccaggagcttcctgctgaagagcctggagcaggtgaggaagatccaggccagcggcagcgtgctgctggagcagctgtgcgccacctacaagctgtgccaccccgaggagctggtgctgctgggccacagcctgggcatccccaaggccagcctgagcggctgcagcagccaggccctgcagcagacccagtgcctgagccagctgcacagcggcctgtgcctgtaccagggcctgctgcaggccctgagcggcatcagccccgccctggcccccaccctggacctgctgcagctggacgtggccaacttcgccaccaccatctggcagcagatggagaacctgggcgtggcccccaccgtgcagcccacccagagcgccatgcccgccttcaccagcgccttccagaggagggccggcggcgtgctggccatcagctacctgcagggcttcctggagaccgccaggctggccctgcaccacctggccgagctcggcagcggcagcgacagggtgtacatccacccctttcacctgggcagcggaagcggaggaagcggaggaagcggaggaagcggcagcggaggaagcggaagcggaggaagcggaggcagcggctctagactgtggccctggccccagtacatccagacctaccacaggaggtacaccctgtaccccaacaacttccagttcaggtaccacgtgagcagcgccgcccaggccggctgcgtggtgctggacgaggccttcaggaggtacaggaacctgctgttcggcagcggcagctggcccaggcccagcttcagcaacaagcagcagaccctgggcaagaacatcctggtggtgagcgtggtgaccgccgagtgcaacgagttccccaacctggagagcgtggagaactacaccctgaccatcaacgacgaccagtgcctgctggccagcgagaccgtgtggggcgccctgaggggcctggagaccttcagccagctggtgtggaagagcgccgagggcaccttcttcatcaacaagaccaagatcaaggacttccccaggttcccccacaggggcgtgctgctggacaccagcaggcactacctgcccctgagcagcatcctggacaccctggacgtgatggcctacaacaagttcaacgtgttccactggcacctggtggacgacagcagcttcccctacgagagcttcaccttccccgagctgaccaggaagggcagcttcaaccccgtgacccacatctacaccgcccaggacgtgaaggaggtgatcgagtacgccaggctgaggggcatcagggtgctggccgagttcgacacccccggccacaccctgagctggggccccggcgcccccggcctgctgaccccctgctacagcggcagccacctgagcggcaccttcggccccgtgaaccccagcctgaacagcacctacgacttcatgagcaccctgttcctggagatcagcagcgtgttccccgacttctacctgcacctgggcggcgacgaggtggacttcacctgctggaagagcaaccccaacatccaggccttcatgaagaagaagggcttcaccgacttcaagcagctggagagcttctacatccagaccctgctggacatcgtgagcgactacgacaagggctacgtggtgtggcaggaggtgttcgacaacaaggtgaaggtgaggcccgacaccatcatccaggtgtggagggaggagatgcccgtggagtacatgctggagatgcaggacatcaccagggccggcttcagggccctgctgagcgccccctggtacctgaacagggtgaagtacggccccgactggaaggacatgtacaaggtggagcccctggccttccacggcacccccgagcagaaggccctggtgatcggcggcgaggcctgcatgtggggcgagtacgtggacagcaccaacctggtgcccaggctgtggcccagggccggcgccgtggccgagaggctgtggagcagcaacctgaccaccaacatcgacttcgccttcaagaggctgagccacttcaggtgcgagctggtgaggaggggcatccaggcccagcccatcagcgtgggctactgcgagcaggagttcgagcagaccggtacccaccaccatcaccatcactaa

SEQ ID NO: 44 - DNA, 993 bp, Artificial Sequence
  Synthetic peptide
cactctgaccctgcccgccgaggggagctgagcgtgtgtgacagtattagtgagtgggtaacggcggcagacaaaaagactgcagtggacatgtcgggcgggacggtcacagtccttgaaaaggtccctgtatcaaaaggccaactgaagcaatacttctacgagaccaagtgcaatcccatgggttacacaaaagaaggctgcaggggcatagacaaaaggcattggaactcccagtgccgaactacccagtcgtacgtgcgggcccttaccatggatagcaaaaagagaattggctggcgattcataaggatagacacttcttgtgtatgtacattgaccattaaaaggggaagaggtaccggcagcgccggcagcgccgccggcagcggcgagttcgtgcccctggtgaccgtgagcgccctgccccccagcctgcccctgcccaggagcttcctgctgaagagcctggagcaggtgaggaagatccaggccagcggcagcgtgctgctggagcagctgtgcgccacctacaagctgtgccaccccgaggagctggtgctgctgggccacagcctgggcatccccaaggccagcctgagcggctgcagcagccaggccctgcagcagacccagtgcctgagccagctgcacagcggcctgtgcctgtaccagggcctgctgcaggccctgagcggcatcagccccgccctggcccccaccctggacctgctgcagctggacgtggccaacttcgccaccaccatctggcagcagatggagaacctgggcgtggcccccaccgtgcagcccacccagagcgccatgcccgccttcaccagcgccttccagaggagggccggcggcgtgctggccatcagctacctgcagggcttcctggagaccgccaggctggccctgcaccacctggccggaagcggcagccacgccatctaccccaggcacggcagccaccaccatcaccatcactaa

SEQ ID NO: 45 - DNA, 357 bp, Artificial Sequence
  Synthetic peptide
cactccgaccctgcccgccgtggggagctgagcgtgtgtgacagtattagcgagtgggtcacagcggcagataaaaagactgcagtggacatgtctggcgggacggtcacagtcctagagaaagtcccggtatccaaaggccaactgaagcagtatttctacgagaccaagtgtaatcccatgggttacaccaaggaaggctgcaggggcatagacaaaaggcactggaactcgcaatgccgaactacccaatcgtatgttcgggcccttactatggatagcaaaaagagaattggctggcgattcataaggatagacacttcctgtgtatgtacattgaccattaaaaggggaaga

SEQ ID NO: 46 - DNA, 1383 bp, Artificial Sequence
  Synthetic peptide
atgaccatccttttccttactatggttatttcatacttcggttgcatgaaggcggcgcccatgaaagaagtaaacgtccacggacaaggcaacttggcctacccaggtgtgcggacccatgggactctggagagcgtgaatgggcccagggcaggttcgagaggtctgacgacgacatcactggctgacacttttgagcacgtcatcgaagagctgctggatgaggaccagaaggttcggcccaacgaagaaaaccataaggacgcggacttgtacacttcccgggtgatgctcagcagtcaagtgcctttggagcctcctctactctttctgctggaggaatacaaaaattacctggatgccgcaaacatgtctatgagggttcggcgccactccgaccctgcccgccgtggggagctgagcgtgtgtgacagtattagcgagtgggtcacagcggcagataaaaagactgcagtggacatgtctggcgggacggtcacagtcctagagaaagtcccggtatccaaaggccaactgaagcagtatttctacgagaccaagtgtaatcccatgggttacaccaaggaaggctgcaggggcatagacaaaaggcactggaactcgcaatgccgaactacccaatcgtatgttcgggcccttactatggatagcaaaaagagaattggctggcgattcataaggatagacacttcctgtgtatgtacattgaccattaaaaggggaagaggtaccggcagcgccggcagcgccgccggcagcggcgagttcgtgcccctggtgaccgtgagcgccctgccccccagcctgcccctgcccaggagcttcctgctgaagagcctggagcaggtgaggaagatccaggccagcggcagcgtgctgctggagcagctgtgcgccacctacaagctgtgccaccccgaggagctggtgctgctgggccacagcctgggcatccccaaggccagcctgagcggctgcagcagccaggccctgcagcagacccagtgcctgagccagctgcacagcggcctgtgcctgtaccagggcctgctgcaggccctgagcggcatcagccccgccctggcccccaccctggacctgctgcagctggacgtggccaacttcgccaccaccatctggcagcagatggagaacctgggcgtggcccccaccgtgcagcccacccagagcgccatgcccgccttcaccagcgccttccagaggagggccggcggcgtgctggccatcagctacctgcagggcttcctggagaccgccaggctggccctgcaccacctggccggaagcggcagccacgccatctaccccaggcacggcagccaccaccatcaccatcactaa

SEQ ID NO: 47 - DNA, 1377 bp, Artificial Sequence
  Synthetic peptide
atgaccatccttttccttactatggttatttcatacttcggttgcatgaaggcggcgcccatgaaagaagtaaacgtccacggacaaggcaacttggcctacccaggtgtgcggacccatgggactctggagagcgtgaatgggcccagggcaggttcgagaggtctgacgacgacatcactggctgacacttttgagcacgtcatcgaagagctgctggatgaggaccagaaggttcggcccaacgaagaaaaccataaggacgcggacttgtacacttcccgggtgatgctcagcagtcaagtgcctttggagcctcctctactctttctgctggaggaatacaaaaattacctggatgccgcaaacatgtctatgagggttcggcgccactccgaccctgcccgccgtggggagctgagcgtgtgtgacagtattagcgagtgggtcacagcggcagataaaaagactgcagtggacatgtctggcgggacggtcacagtcctagagaaagtcccggtatccaaaggccaactgaagcagtatttctacgagaccaagtgtaatcccatgggttacaccaaggaaggctgcaggggcatagacaaaaggcactggaactcgcaatgccgaactacccaatcgtatgttcgggcccttactatggatagcaaaaagagaattggctggcgattcataaggatagacacttcctgtgtatgtacattgaccattaaaaggggaagaggtaccggcagcgccggcagcgccgccggcagcggcgagttcgccacccctctgggccctgcctccagcctgccccagtccttcctgctgaagtgcctggaacaggtgcgcaagatccagggcgacggcgctgccctgcaggaaaagctctgcgccacctacaagctgtgccaccccgaggaactggtgctgctgggacacagcctgggaatcccttgggcccctctgtcctcctgccctagccaggctctgcagctggccggatgcctgtcccagctgcactccggcctgttcctctaccagggactgctgcaggctctcgaaggcatctcccccgagctgggccctaccctggacacactgcagctggacgtggccgacttcgccaccaccatctggcagcagatggaagaactgggcatggccccagctctgcagcctacccagggcgccatgcctgccttcgcctccgccttccagagaagggctggaggagtgctggtggcctcccacctgcagtcattcctcgaagtgtcctaccgcgtgctgaggcacctggcccagcccggaagcggcagccacgccatctaccccaggcacggcagccaccaccatcaccatcactaatga

SEQ ID NO: 48 - DNA, 1377 bp, Artificial Sequence
  Synthetic peptide
atgaccatccttttccttactatggttatttcatactttggttgcatgaaggctgcccccatgaaagaagcaaacatccgaggacaaggtggcttggcctacccaggtgtgcggacccatgggactctggagagcgtgaatgggcccaaggcaggttcaagaggcttgacatcattggctgacactttcgaacacgtgatagaagagctgttggatgaggaccagaaagttcggcccaatgaagaaaacaataaggacgcagacttgtacacgtccagggtgatgctcagtagtcaagtgcctttggagcctcctcttctctttctgctggaggaatacaaaaattacctagatgctgcaaacatgtccatgagggtccggcgccactctgaccctgcccgccgaggggagctgagcgtgtgtgacagtattagtgagtgggtaacggcggcagacaaaaagactgcagtggacatgtcgggcgggacggtcacagtccttgaaaaggtccctgtatcaaaaggccaactgaagcaatacttctacgagaccaagtgcaatcccatgggttacacaaaagaaggctgcaggggcatagacaaaaggcattggaactcccagtgccgaactacccagtcgtacgtgcgggcccttaccatggatagcaaaaagagaattggctggcgattcataaggatagacacttcttgtgtatgtacattgaccattaaaaggggaagaggtaccggcagcgccggcagcgccgccggcagcggcgagttcgtgcccctggtgaccgtgagcgccctgccccccagcctgcccctgcccaggagcttcctgctgaagagcctggagcaggtgaggaagatccaggccagcggcagcgtgctgctggagcagctgtgcgccacctacaagctgtgccaccccgaggagctggtgctgctgggccacagcctgggcatccccaaggccagcctgagcggctgcagcagccaggccctgcagcagacccagtgcctgagccagctgcacagcggcctgtgcctgtaccagggcctgctgcaggccctgagcggcatcagccccgccctggcccccaccctggacctgctgcagctggacgtggccaacttcgccaccaccatctggcagcagatggagaacctgggcgtggcccccaccgtgcagcccacccagagcgccatgcccgccttcaccagcgccttccagaggagggccggcggcgtgctggccatcagctacctgcagggcttcctggagaccgccaggctggccctgcaccacctggccggaagcggcagccacgccatctaccccaggcacggcagccaccaccatcaccatcactaa

SEQ ID NO: 49 - protein, 458 aa, Artificial Sequence
  Synthetic peptide
MTILFLTMVISYFGCMKAAPMKEANIRGQGGLAYPGVRTHGTLESVNGPKAGSRGLTSLADTFEHVIEELLDEDQKVRPNEENNKDADLYTSRVMLSSQVPLEPPLLFLLEEYKNYLDAANMSMRVRRHSDPARRGELSVCDSISEWVTAADKKTAVDMSGGTVTVLEKVPVSKGQLKQYFYETKCNPMGYTKEGCRGIDKRHWNSQCRTTQSYVRALTMDSKKRIGWRFIRIDTSCVCTLTIKRGRGTGSAGSAAGSGEFVPLVTVSALPPSLPLPRSFLLKSLEQVRKIQASGSVLLEQLCATYKLCHPEELVLLGHSLGIPKASLSGCSSQALQQTQCLSQLHSGLCLYQGLLQALSGISPALAPTLDLLQLDVANFATTIWQQMENLGVAPTVQPTQSAMPAFTSAFQRRAGGVLAISYLQGFLETARLALHHLAGSGSHAIYPRHGSHHHHHH

SEQ ID NO: 50 - protein, 460 aa, Artificial Sequence
  Synthetic peptide
MTILFLTMVISYFGCMKAAPMKEVNVHGQGNLAYPGVRTHGTLESVNGPRAGSRGLTTTSLADTFEHVIEELLDEDQKVRPNEENHKDADLYTSRVMLSSQVPLEPPLLFLLEEYKNYLDAANMSMRVRRHSDPARRGELSVCDSISEWVTAADKKTAVDMSGGTVTVLEKVPVSKGQLKQYFYETKCNPMGYTKEGCRGIDKRHWNSQCRTTQSYVRALTMDSKKRIGWRFIRIDTSCVCTLTIKRGRGTGSAGSAAGSGEFVPLVTVSALPPSLPLPRSFLLKSLEQVRKIQASGSVLLEQLCATYKLCHPEELVLLGHSLGIPKASLSGCSSQALQQTQCLSQLHSGLCLYQGLLQALSGISPALAPTLDLLQLDVANFATTIWQQMENLGVAPTVQPTQSAMPAFTSAFQRRAGGVLAISYLQGFLETARLALHHLAGSGSHAIYPRHGSHHHHHH

SEQ ID NO: 51 - protein, 6 aa, Artificial Sequence
  Synthetic peptide
EAAAAK